(19) United States
(12) Patent Application Publication
(10) Pub. No.: US 2006/0160811 A1
(43) Pub. Date: Jul. 20, 2006

(54) Aryl-condensed 3-arylpridine compounds and use thereof for controlling pathogenic fungi

(76) Inventors: Oliver Wagner, NEUSTADT (DE); Thomas Grote, Wachenheim (DE); Carsten Blettner, Mannheim (DE); Markus Gewehr, Kastellaun (DE); Wassilios Grammenos, Ludwigshafen (DE); Andreas Gypser, Mannheim (DE); Bernd Muller, Frankenthal (DE); Joachim Rheinheimer, Ludwigshafen (DE); Peter Schafer, Ottersheim (DE); Frank Schieweck, Hessheim (DE); Anja Schwogler, Mannheim (DE); Jordi Tormo i Blasco, Laudenbach (DE); Alan Akers, Brumath (GB); John-Bryan Speakman, Bobenheim (DE); Michael Rack, Heidelberg (DE); Reinhard Stierl, Freinsheim (DE); Maria Scherer, Landau (DE); Siegfried Strathmann, Limburgerhof (DE); Ulrich Schofl, Bruhl (DE)

Correspondence Address: BIRCH STEWART KOLASCH & BIRCH, PO BOX 747, FALLS CHURCH, VA, 22040-0747, US

(21) Appl. No.: 10/563,222

(22) PCT Filed: Jul. 15, 2004
(86) PCT No.: PCT/EP04/07924

§ 371 Date: Jan. 4, 2006

(30) Foreign Application Priority Data
Jul. 18, 2003 (DE) 103 32 790.8

(51) Int. Cl. C07D 487/02; C07D 471/02; A61K 31/53; A61K 31/519

(52) U.S. Cl. 514/243; 514/249; 514/264.1; 514/300; 544/184; 544/279; 544/300; 546/122

(57) ABSTRACT

The present invention relates to bicyclic compounds of general formula I, 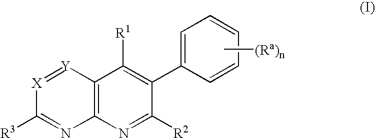 in which
X, Y independently of one another are N or C—R⁴; n is 1, 2, 3, 4 or 5;
R^{a} is halogen, cyano, C₁-C₆-alkyl, C₁-C₆-alkoxy, C₁-C₆-haloalkyl, C₁-C₆-haloalkoxy, C₂-C₆-alkenyl, C₂-C₆-alkenyloxy or C(O)R⁵;
R¹ is halogen, cyano, C₁-C₆-alkyl, C₁-C₆-haloalkyl, C₂-C₆-alkenyl, C₂-C₆-alkynyl, C₃-C₈-cycloalkyl which is optionally mono- or polysubstituted by alkyl and/or halogen, C₅-C₈-cycloalkenyl which is optionally mono- or polysubstituted by alkyl and/or halogen, OR⁶, SR⁶ or NR⁷R⁸;
R² is halogen, cyano, C₁-C₆-alkyl, C₁-C₆-haloalkyl, C₂-C₆-alkenyl, C₂-C₆-alkynyl, C₃-C₈-cycloalkyl which is optionally mono- or polysubstituted by alkyl and/or halogen, C₅-C₈-cycloalkenyl which is optionally mono- or polysubstituted by alkyl and/or halogen, OR⁶, SR⁶ or NR⁷R⁸;
R³ is hydrogen, C₁-C₆-alkyl, C₁-C₆-haloalkyl or C₃-C₆-cycloalkyl which is optionally mono- or polysubstituted by alkyl and/or halogen; and the agriculturally acceptable salts of compounds I, plant protection agents, containing at least one compound of general formula I and/or one agriculturally-acceptable salt of I and at least one liquid or solid carrier substance, as well as a method for controlling phytopathogenic fungi.

The present invention relates to novel aryl-fused 3-arylpyridine compounds and to their use for controlling harmful fungi, and also to crop protection compositions comprising such compounds as active component.

EP-A 71792, U.S. Pat. No. 5,994,360, EP-A 550113 and WO 02/48151 describe fungicidally active pyrazolo[1,5-a]pyrimidines and triazolo[1,5a]pyrimidines which carry an optionally substituted phenyl group in the 5-position of the pyrimidine ring. Imidazolo[1,2-a]pyrimidines having fungicidal action are known from WO 03/022850.

In principle, there is a constant demand for novel fungicidally active compounds to widen the activity spectrum and to prevent a possible development of resistance against known fungicides. Novel active compounds should kill the harmful fungi at application rates which are as low as possible and reduce or, even better, prevent their re-establishment. Moreover, the active compounds should be well tolerated by useful plants, i.e. they should cause little, if any, damage to the useful plants.

U.S. Pat. No. 5,801,183 and WO 96/22990 describe 2,4-dihydroxy-1,8-naphthyridines which carry an optionally substituted phenyl radical in the 3-position, as aza analogs of glycine/NMDA receptor antagonists.

The compounds 4-hydroxy-3-(o-methoxyphenyl)-1,8-naphthyridin-2-(1H)-one and 2,4-dichloro-3-(o-methoxyphenyl)-1,8-naphthyridine are known from J. of Heterocyclic Chemistry, 30, 1993, 909-912, and 4-hydroxy-3-(4-methoxyphenyl)-1,8-naphthyridin-2-(1H)-one, 4-hydroxy-3-(4-methylphenyl)-1,8-naphthyridin-2-(1H)-one, 4-hydroxy-3-(3-methylphenyl)-1,8-naphthyridin-2-(1H)-one and 4-hydroxy-3-(2-methylphenyl)-1,8-naphthyridin-2-(1H)-one are known from Chem. Ber. 111, 2813-2824 (1978).

With a view to a possible development of resistance and to widening of the activity spectrum, it is of fundamental interest to provide novel active compounds.

Accordingly, it is an object of the present invention to provide novel compounds having good fungicidal activity, in particular at low application rates, and/or good compatibility with useful plants. This object is achieved by bicyclic, i.e. aryl-fused, 3-arylpyridine compounds of the formula I 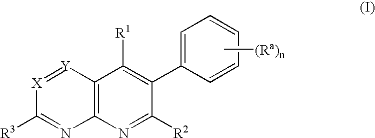
in which
X, Y independently of one another are N or C—R⁴;
n is 1, 2, 3, 4 or 5;
R^{a} is halogen, cyano, C₁-C₆-alkyl, C₁-C₆-alkoxy, C₁-C₆-haloalkyl, C₁-C₆-haloalkoxy, C₂-C₆-alkenyl, C₂-C₆-alkenyloxy or C(O)R⁵;
R¹ is halogen, cyano, C₁-C₆-alkyl, C₁-C₆-haloalkyl, C₂-C₆-alkenyl, C₂-C₆-alkynyl, C₃-C₈-cycloalkyl which is optionally mono- or polysubstituted by alkyl and/or halogen, C₅-C₈-cycloalkenyl which is optionally mono- or polysubstituted by alkyl and/or halogen, OR⁶, SR⁶ or NR⁷R⁸;
R² is halogen, cyano, C₁-C₆-alkyl, C₁-C₆-haloalkyl, C₂-C₆-alkenyl, C₂-C₆-alkynyl, C₃-C₈-cycloalkyl which is optionally mono- or polysubstituted by alkyl and/or halogen, C₅-C₈-cycloalkenyl which is optionally mono- or polysubstituted by alkyl and/or halogen, OR⁶, SR⁶ or NR⁷R⁸;
R³ is hydrogen, C₁-C₆-alkyl, C₁-C₆-haloalkyl or C₃-C₆-cycloalkyl which is optionally mono- or polysubstituted by alkyl and/or halogen;
R⁴ is hydrogen, halogen, C₁-C₆-alkyl, C₁-C₆-haloalkyl or C₃-C₆-cycloalkyl which is optionally mono- or polysubstituted by alkyl and/or halogen;
R⁵ is hydrogen, OH, C₁-C₆-alkyl, C₁-C₆-alkoxy, C₁-C₆-haloalkyl, C₁-C₆-haloalkoxy, C₂-C₆-alkenyl, C₁-C₆-alkylamino or di-C₁-C₆-alkylamino, piperidin-1-yl, pyrrolidin-1-yl or morpholin-4-yl;
R⁶ is hydrogen, C₁-C₆-alkyl, C₁-C₆-haloalkyl, phenyl-C₁-C₄-alkyl where phenyl may be mono- or polysubstituted by halogen, alkyl or alkoxy, C₂-C₆-alkenyl or COR⁹;
R⁷, R⁸ independently of one another are hydrogen, C₁-C₁₀-alkyl, C₂-C₁₀-alkenyl, C₄-C₁₀-alkadienyl, C₂-C₁₀-alkynyl, C₃-C₈-cycloalkyl, C₅-C₈-cycloalkenyl, C₅-C₁₀-bicycloalkyl, phenyl, phenyl-C₁-C₄-alkyl, naphthyl,
   a 5- or 6-membered saturated or partially unsaturated heterocycle which may have 1, 2 or 3 heteroatoms selected from the group consisting of N, O and S as ring members, or
   a 5- or 6-membered aromatic heterocycle which may have 1, 2 or 3 heteroatoms selected from the group consisting of N, O and S as ring members,
   where the radicals mentioned as R⁷, R⁸ may be partially or fully halogenated and/or may have 1, 2 or 3 radicals R^{b}, where
      R^{b} is selected from the group consisting of cyano, nitro, OH, C₁-C₆-alkyl, C₁-C₆-alkoxy, C₁-C₆-haloalkyl, C₁-C₆-haloalkoxy, C₁-C₆-alkylthio, C₂-C₆-alkenyl, C₂-C₆-alkenyloxy, C₂-C₆-alkynyl, C₂-C₆-alkynyloxy, C₁-C₆-alkylamino, di-C₁-C₆-alkylamino, piperidin-1-yl, pyrrolidin-1-yl or morpholin-4-yl;
R⁷ and R⁸ together with the nitrogen atom to which they are attached may also form a 5-, 6- or 7-membered saturated or unsaturated heterocycle which may have 1, 2, 3 or 4 further heteroatoms selected from the group consisting of O, S, N and NR¹⁰ as ring members and may be partially or fully halogenated and which may have 1, 2 or 3 radicals R^{b}; and
R⁹, R¹⁰ independently of one another are hydrogen or C₁-C₆-alkyl; and the agriculturally acceptable salts of compounds I.

Accordingly, the present invention provides the use of the bicyclic compounds of the formula I and their agriculturally acceptable salts for controlling phytopathogenic fungi (=harmful fungi), and a method for controlling phytopathogenic harmful fungi which comprises treating the fungi or the materials, plants, the soil or seed to be protected against fungal attack with an effective amount of the compound of the formula I and/or with an agriculturally acceptable salt of I.

Except for the 1,8-naphthyridines mentioned in U.S. Pat. No. 5,801,183, in WO 96/22990, in J. of Heterocyclic Chemistry, 30, 1993, 909-912 and in Chem. Ber. 111, 2813-2824 (1978), the compounds I are novel. Accordingly, the present invention also relates to bicyclic compounds of the formula I and agriculturally acceptable salts thereof, except for:

compounds of the formula I in which R¹ is OH if Y and X are simultaneously each C—R⁴; and also
2,4-dichloro-3-(o-methoxyphenyl)-1,8-naphthyridine.

The present invention furthermore provides a composition for controlling harmful fungi, comprising at least one compound of the formula I and/or an agriculturally acceptable salt thereof and at least one liquid or solid carrier.

Depending on the substitution pattern, the compounds of the formula I may have one or more centers of chirality, in which case they are present as pure enantiomers or diastereomers or as mixtures of enantiomers or diastereomers. The invention provides both the pure enantiomers or diastereomers and their mixtures. The invention also provides tautomers of compounds of the formula I.

Suitable agriculturally useful salts are especially the salts of those cations or the acid addition salts of those acids whose cations and anions, respectively, have no negative effect on the fungicidal action of the compounds I. Thus, suitable cations are in particular the ions of the alkali metals, preferably sodium and potassium, of the alkaline earth metals, preferably calcium, magnesium and barium, and of the transition metals, preferably manganese, copper, zinc and iron, and also the ammonium ion which, if desired, may carry one to four C₁-C₄-alkyl substituents and/or one phenyl or benzyl substituent, preferably diisopropylammonium, tetramethylammonium, tetrabutylammonium, trimethylbenzylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogensulfate, sulfate, dihydrogenphosphate, hydrogenphosphate, phosphate, nitrate, hydrogencarbonate, carbonate, hexafluorosilicate, hexafluorophosphate, benzoate, and the anions of C₁-C₄-alkanoic acids, preferably formate, acetate, propionate and butyrate. They can be formed by reacting I with an acid of the corresponding anion, preferably hydrochloric acid, hydrobromic acid, sulfuric acid, phosphoric acid or nitric acid.

In the definitions of the variables given in the formulae above, collective terms are used which are generally representative for the respective substituents. The term Cₙ-Cₘ denotes the number of carbon atoms possible in each case in the respective substituent or substituent moiety:

halogen: fluorine, chlorine, bromine and iodine;

alkyl and all alkyl moieties in alkoxy, alkylthio, alkylamino and dialkylamino: saturated straight-chain or branched hydrocarbon radicals having 1 to 4, to 6, to 8 or to 10 carbon atoms, for example C₁-C₆-alkyl, such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylproyl, 1-ethyl-1-methylpropyl and 1-ethyl-2-methylpropyl;
   haloalkyl: straight-chain or branched alkyl groups having 1 to 4 or to 6 carbon atoms (as mentioned above), where some or all of the hydrogen atoms in these groups may be replaced by halogen atoms as mentioned above, for example C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl and 1,1,1-trifluoroprop-2-yl;
   phenyl-C₁-C₄-alkyl: a C₁-C₄-alkyl group as mentioned above which is substituted by phenyl, for example benzyl, 1-phenylethyl, 2-phenylethyl, 1-phenylprop-1-yl, 2-phenylprop-1-yl, 3-phenylprop-1-yl, 1-phenylbut-1-yl, 2-phenylbut-1-yl, 3-phenylbut-1-yl, 4-phenylbut-1-yl, 1-phenylbut-2-yl, 2-phenylbut-2-yl, 3-phenylbut-2-yl, 4-phenylbut-2-yl, 1-(phenylmeth)eth-1-yl, 1-(phenylmethyl)-1-(methyl)eth-1-yl or -(phenylmethyl)-1-(methyl)prop-1-yl; preferably benzyl;
   phenyl-C₁-C₄-alkyl which is optionally mono- or polysubstituted by halogen, alkoxy or alkyl: a phenyl-substituted C₁-C₄-alkyl group, where the phenyl group unsubstituted or may carry 1, 2, 3 or 4, preferably 1, substituent(s) selected from the group consisting of fluorine, chlorine, bromine, C₁-C₆-alkoxy and C₁-C₆-alkyl, for example p-bromophenylmethyl, p-chlorophenylmethyl, p-methylphenylmethyl, p-methylphenylmethyl, p-methoxyphenylmethyl, p-methoxyphenylethyl;
   alkenyl: monounsaturated straight-chain or branched hydrocarbon radicals having 2 to 4, to 6, to 8 or to 10 carbon atoms and a double bond in any position, for example C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;
   alkadienyl: diunsaturated straight-chain or branched hydrocarbon radicals having 4 to 10 carbon atoms and two double bonds in any position, for example 1,3-butadienyl, 1-methyl-1,3-butadienyl, 2-methyl-1,3-butadienyl, penta-1,3-dien-1-yl, hexa-1,4-dien-1-yl, hexa-1,4-dien-3-yl, hexa-1,4-dien-6-yl, hexa-1,5-dien-1-yl, hexa-1,5-dien-3-yl, hexa-1,5-dien-4-yl, hepta-1,4-dien-1-yl, hepta-1,4-dien-3-yl, hepta-1,4-dien-6-yl, hepta-1,4-dien-7-yl, hepta-1,5-dien-1-yl, hepta-1,5-dien-3-yl, hepta-1,5-dien-4-yl, hepta-1,5-dien-7-yl, hepta-1,6-dien-1-yl, hepta-1,6-dien-3-yl, hepta-1,6-dien-4-yl, hepta-1,6-dien-5-yl, hepta-1,6-dien-2-yl, octa-1,4-dien-1-yl, octa-1,4-dien-2-yl, octa-1,4-dien-3-yl, octa-1,4-dien-6-yl, octa-1,4-dien-7-yl, octa-1,5-dien-1-yl, octa-1,5-dien-3-yl, octa-1,5-dien-4-yl, octa-1,5-dien-7-yl, octa-1,6-dien-1-yl, octa-1,6-dien-3-yl, octa-1,6-dien-4-yl, octa-1,6-dien-5-yl, octa-1,6-dien-2-yl, deca-1,4-dienyl, deca-1,5-dienyl, deca-1,6-dienyl, deca-1,7-dienyl, deca-1,8-dienyl, deca-2,5-dienyl, deca-2,6-dienyl, deca-2,7-dienyl, deca-2,8-dienyl and the like;
   alkynyl: straight-chain or branched hydrocarbon groups having 2 to 4, 2 to 6, 2 to 8 or 2 to 10 carbon atoms and a triple bond in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-ethyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;
   cycloalkyl: monocyclic saturated hydrocarbon groups having 3 to 8, preferably to 6, carbon ring members, such as cyclopropyl, cyclobutyl, cyclopentyl and cyclohexyl;
   cycloalkyl which is optionally mono- or polysubstituted by halogen or alkyl: a cycloalkyl group as mentioned above which is unsubstituted or carries 1, 2, 3 or 4, preferably 1, substituent(s), where the substituent(s) is/are selected from the group consisting of fluorine, chlorine, bromine and C₁-C₆-alkyl, for example 4-chlorocyclohexyl, 4-bromocyclohexyl, 4-methoxycyclohexyl, 4-ethylcyclohexyl, 2-chlorocyclopropyl, 2-fluorocyclopropyl, 1-chlorocyclopropyl, 1-fluorocyclopropyl;
   cycloalkenyl: monocyclic monounsaturated hydrocarbon groups having 5 to 8, preferably 6, carbon ring members, such as cyclopenten-1-yl, cyclopenten-3-yl, cyclohexen-1-yl, cyclohexen-3-yl and cyclohexen-4-yl;
   cycloalkenyl which is optionally mono- or polysubstituted by halogen or alkyl: a cycloalkenyl group as mentioned above which is unsubstituted or carries 1, 2, 3 or 4, preferably 1, substituent(s), where the substituent(s) is/are selected from the group consisting of fluorine, chlorine, bromine and C₁-C₆-alkyl, for example 4-chlorocyclohexen-1-yl, 4-bromocyclohexen-1-yl, 4-methylcyclohexen-1-yl, 4-ethylcyclohexen-1-yl, 4-chlorocyclohexen-3-yl, 4-bromocyclohexen-3-yl, 4-methylcyclohexen-3-yl, 4-ethylcyclohexen-3-yl;
   bicycloalkyl: a bicyclic hydrocarbon radical having 5 to 10 carbon atoms, such as bicyclo[2.2.1]hept-1-yl, bicyclo[2.2.1]hept-2-yl, bicyclo[2.2.1]hept-7-yl, bicyclo[2.2.2]oct-1-yl, bicyclo[2.2.2]oct-2-yl, bicyclo[3.3.0]octyl and bicyclo[4.4.0]decyl;
   C₁-C₄-alkoxy: an alkyl group having 1 to 4 carbon atoms which is attached via oxygen: for example methoxy, ethoxy, n-propoxy, 1-methylethoxy, butoxy, 1-methylpropoxy, 2-methylpropoxy or 1,1-dimethylethoxy;
   C₁-C₆-alkoxy: C₁-C₄-alkoxy as mentioned above, and also, for example, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy;
   C₁-C₄-haloalkoxy: a C₁-C₄-alkoxy radical as mentioned above which is partially or fully substituted by fluorine, chlorine, bromine and/or iodine, preferably by fluorine, i.e., for example, OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, OC₂F₅, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy;
   C₁-C₆-haloalkoxy: C₁-C₄-haloalkoxy as mentioned above, and also, for example, 5-fluoropentoxy, 5-chloropentoxy, 5-bromopentoxy, 5-iodopentoxy, undecafluoropentoxy, 6-fluorohexoxy, 6-chlorohexoxy, 6-bromohexoxy, 6-iodohexoxy or dodecafluorohexoxy;
   alkenyloxy: alkenyl as mentioned above which is attached via an oxygen atom, for example C₂-C₆-alkenyloxy, such as vinyloxy, 1-propenyloxy, 2-propenyloxy, 1-methylethenyloxy, 1-butenyloxy, 2-butenyloxy, 3-butenyloxy, 1-methyl-1-propenyloxy, 2-methyl-1-propenyloxy, 1-methyl-2-propenyloxy, 2-methyl-2-propenyloxy, 1-pentenyloxy, 2-pentenyloxy, 3-pentenyloxy, 4-pentenyloxy, 1-methyl-1-butenyloxy, 2-methyl-1-butenyloxy, 3-methyl-1-butenyloxy, 1-methyl-2-butenyloxy, 2-methyl-2-butenyloxy, 3-methyl-2-butenyloxy, 1-methyl-3-butenyloxy, 2-methyl-3-butenyloxy, 3-methyl-3-butenyl, 1,1-dimethyl-2-propenyloxy, 1,2-dimethyl-1-propenyloxy, 1,2-dimethyl-2-propenyloxy, 1-ethyl-1-propenyloxy, 1-ethyl-2-propenyloxy, 1-hexenyloxy, 2-hexenyloxy, 3-hexenyloxy, 4-hexenyloxy, 5-hexenyloxy, 1-methyl-1-pentenyloxy, 2-methyl-1-pentenyloxy, 3-methyl-1-pentenyloxy, 4-methyl-1-pentenyloxy, 1-methyl-2-pentenyloxy, 2-methyl-2-pentenyloxy, 3-methyl-2-pentenyloxy, 4-methyl-2-pentenyloxy, 1-methyl-3-pentenyloxy, 2-methyl-3-pentenyloxy, 3-methyl-3-pentenyloxy, 4-methyl-3-pentenyloxy, 1-methyl-4-pentenyloxy, 2-methyl-4-pentenyloxy, 3-methyl-4-pentenyloxy, 4-methyl-4-pentenyloxy, 1,1-dimethyl-2-butenyloxy, 1,1-dimethyl-3-butenyloxy, 1,2-dimethyl-1-butenyloxy, 1,2-dimethyl-2-butenyloxy, 1,2-dimethyl-3-butenyloxy, 1,3-dimethyl-1-butenyloxy, 1,3-dimethyl-2-butenyloxy, 1,3-dimethyl-3-butenyloxy, 2,2-dimethyl-3-butenyloxy, 2,3-dimethyl-1-butenyloxy, 2,3-dimethyl-2-butenyloxy, 2,3-dimethyl-3-butenyloxy, 3,3-dimethyl-1-butenyloxy, 3,3-dimethyl-2-butenyloxy, 1-ethyl-1-butenyloxy, 1-ethyl-2-butenyloxy, 1-ethyl-3-butenyloxy, 2-ethyl-1-butenyloxy, 2-ethyl-2-butenyloxy, 2-ethyl-3-butenyloxy, 1,1,2-trimethyl-2-propenyloxy, 1-ethyl-1-methyl-2-propenyloxy, 1-ethyl-2-methyl-1-propenyloxy and 1-ethyl-2-methyl-2-propenyloxy;
   alkynyloxy: alkynyl as mentioned above which is attached via an oxygen atom, for example C₃-C₆-alkynyloxy, such as 2-propynyloxy, 2-butynyloxy, 3-butynyloxy, 1-methyl-2-propynyloxy, 2-pentynyloxy, 3-pentynyloxy, 4-pentynyloxy, 1-methyl-2-butynyloxy, 1-methyl-3-butynyloxy, 2-methyl-3-butynyloxy, 1-ethyl-2-propynyloxy, 2-hexynyloxy, 3-hexynyloxy, 4-hexynyloxy, 5-hexynyloxy, 1-methyl-2-pentynyloxy, 1-methyl-3-pentynyloxy and the like;
   a five- or six-membered saturated or partially unsaturated heterocycle which comprises one, two or three heteroatoms from the group consisting of oxygen, nitrogen and sulfur: for example mono- and bicyclic heterocycles (heterocyclyl) which, in addition to carbon ring members, comprise one to three nitrogen atoms and/or one oxygen or sulfur atom or one or two oxygen and/or sulfur atoms, for example 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-diyydrooxazol-3-yl, 3,4-dihydrooxazol-4-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl;
   a five- or six-membered aromatic heterocycle which comprises one, two or three heteroatoms from the group consisting of oxygen, nitrogen or sulfur: mono- or bicyclic heteroaryl, for example 5-membered heteroaryl which is attached via carbon and comprises one to three nitrogen atoms or one or two nitrogen atoms and one sulfur or oxygen atom as ring members, such as 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl; 5-membered heteroaryl which is attached via nitrogen and contains one to three nitrogen atoms as ring members, such as pyrrol-1-yl, pyrazol-1-yl, imidazol-1-yl, 1,2,3-triazol-1-yl and 1,2,4-triazol-1-yl; 6-membered heteroaryl which comprises one to three nitrogen atoms one to three nitrogen atoms as ring members, such as pyridin-2-yl, pyridin-3-yl, pyridin-4-yl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

A first embodiment of the present invention relates to compounds of the formula I in which X and Y are each C—R⁴, where the radicals R⁴ may in each case be identical or different. Hereinbelow, these compounds are referred to as compounds I.a.

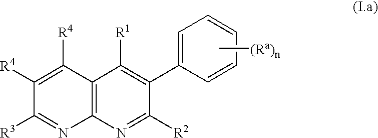

A further preferred embodiment of the present invention relates to compounds of the formula I in which X is C—R⁴ and Y is N. Hereinbelow, these compounds are referred to as compounds I.b.

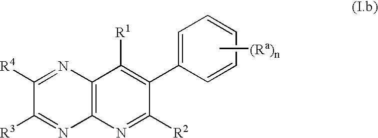

A further preferred embodiment of the present invention relates to compounds of the formula I in which X is N and Y is C—R⁴. Hereinbelow, these compounds are referred to as compounds I.c.

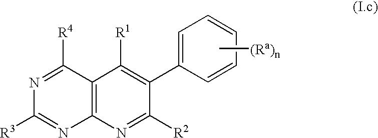

In the formulae of the formula I.a, I.b and I.c, the variables R^{a}, n, R¹, R², R³ and R⁴ have the meanings given above, in particular the meanings given below as being preferred.

With a view to the use of the compounds I according to the invention as fungicides, the variables n, R^{a}, R¹, R², R³ and R⁴ independently of one another and preferably in combination have the following meanings:
n is 2, 3, 4 or 5, in particular 2 or 3;
R^{a} is halogen, in particular fluorine or chlorine, C₁-C₄-alkyl, in particular methyl, C₁-C₄-alkoxy, in particular methoxy, C₁-C₂-fluoroalkyl, in particular difluoromethyl and trifluoromethyl, C₁-C₂-fluoroalkoxy, in particular difluoromethoxy and trifluoromethoxy, C₁-C₄-alkoxycarbonyl, in particular methoxycarbonyl and cyano. Particularly preferably, R^{a} is selected from the group consisting of halogen, especially fluorine or chlorine, C₁-C₄-alkyl, especially methyl, and C₁-C₄-alkoxy, especially methoxy;
R¹ is halogen, especially chlorine, hydroxyl or a group NR⁷R⁸;
R² is halogen, especially chlorine, hydroxyl, C₁-C₆-alkyl, especially methyl, C₁-C₆-haloalkyl or a group NR⁷R⁸;
R³ is hydrogen, C₁-C₆-alkyl, preferably C₁-C₃-alkyl, C₁-C₆-haloalkyl, preferably C₁-C₃-haloalkyl, and particularly preferably hydrogen;
R⁴ is hydrogen, halogen, C₁-C₆-alkyl, preferably C₁-C₃-alkyl, C₁-C₆-haloalkyl, preferably C₁-C₃-haloalkyl, particularly preferably hydrogen.

If R¹ is halogen, especially chlorine, R² is preferably halogen, especially chlorine, C₁-C₆-alkyl, especially methyl, C₁-C₆-haloalkyl or a group NR⁷R⁸.

If R¹ is hydroxyl, R² is preferably hydroxyl, C₁-C₆-alkyl or C₁-C₆-haloalkyl.

If R¹ is a group NR⁷R⁸, R² is preferably selected from halogen, especially chlorine, C₁-C₆-alkyl, especially methyl, and C₁-C₆-haloalkyl.

If R¹ is a group NR⁷R⁸, preferably at least one of the radicals R⁷, R⁸ is different from hydrogen. In particular, R⁷ is C₁-C₆-alkyl, C₃-C₈-cycloalkyl which is optionally mono- or polysubstituted by alkyl, is C₁-C₆-haloalkyl, phenyl-C₁-C₄-alkyl, C₂-C₆-alkenyl or C₂-C₆-alkynyl. R⁸ is in particular hydrogen, C₁-C₆-alkyl or C₂-C₆-alkenyl and very particularly preferably hydrogen or C₁-C₄-alkyl.

The preferred groups NR⁷R⁸ include those which are a saturated or partially unsaturated heterocyclic radical which, in addition to the nitrogen atom, may have a further heteroatom selected from the group consisting of O, S and NR¹⁰ as ring member and which may have one or two substituents selected from the group consisting of halogen, hydroxyl, C₁-C₆-alkyl and C₁-C₆-haloalkyl. Preferably, the heterocyclic radical has 5 to 7 atoms as ring members. Examples of such heterocyclic radicals are pyrrolidine, piperidine, morpholine, tetrahydropyridine, for example 1,2,3,6-tetrahydropyridine, piperazine and azepane, which may be substituted in the manner mentioned above.

If R² is a group NR⁷R⁸, preferably at least one of the radicals R⁷, R⁸ is different from hydrogen. In particular, R⁷ has the meanings mentioned above as being preferred.

Preferred meanings of R^{a} include halogen, especially F or Cl, trifluoromethyl, CN, C₁-C₄-alkoxy, C₁-C₄-alkoxycarbonyl, in particular methoxycarbonyl.

With a view to the use of the compounds I according to the invention as fungicides,
the radical 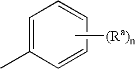
is preferably a radical of the formula 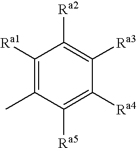
in which R^{a1} has the meanings mentioned above for R^{a} and the radicals R^{a2}, R^{a3}, R^{a4} and R^{a5} have the meanings given for R^{a} or are hydrogen. In particular:
R^{a1} is fluorine, chlorine, trifluoromethyl or methyl;
R^{a2} is hydrogen or fluorine;
R^{a3} is hydrogen, fluorine, chlorine, cyano, C₁-C₄-alkyl, especially methyl, C₁-C₄-alkoxy, especially methoxy, or C₁-C₄-alkoxycarbonyl, especially methoxycarbonyl;
R^{a4} is hydrogen, chlorine or fluorine;
R^{a5} is hydrogen, fluorine, chlorine or C₁-C₄-alkyl, especially methyl, or C₁-C₄-alkoxy, especially methoxy.

Here, preferably at least one of the radicals R^{a3} or R^{a5} is different from hydrogen. Preferably, at least one and particularly preferably both radicals R^{a2}, R^{a4} are hydrogen.

A preferred embodiment of the compounds I.b according to the invention is that in which R² is halogen, cyano, C₁-C₆-alkyl, C₁-C₆-haloalkyl, C₂-C₆-alkenyl, C₂-C₆-alkynyl, C₃-C₈-cycloalkyl which is optionally mono- or polysubstituted by alkyl and/or halogen, C₅-C₈-cycloalkenyl which is optionally mono- or polysubstituted by alkyl and/or halogen or NR⁷R⁸ in which R⁷ and R⁸ are each different from hydrogen.

Another preferred embodiment of the compounds I.a and I.c according to the invention relates to those in which R² is halogen, cyano, C₁-C₆-alkyl, C₁-C₆-haloalkyl, C₂-C₆-alkenyl, C₂-C₆-alkynyl, C₃-C₈-cycloalkyl which is optionally mono- or polysubstituted by alkyl and/or halogen, C₅-C₈-cycloalkenyl which is optionally mono- or polysubstituted by alkyl and/or halogen, OR⁵, SR⁶ or N R⁷R⁸ where R⁶, R⁷ and R⁸ have the meanings mentioned above and in particular the preferred meanings.

Otherwise, the variables R³, R⁴, R⁵ and R⁶ independently of one another and preferably in combination with the preferred meanings of the variables, n, R^{a}, R¹ and R² have the following meanings:
R³ is hydrogen, C₁-C₆-alkyl, preferably C₁-C₃-alkyl, C₁-C₆-haloalkyl, preferably C₁-C₃-haloalkyl, and particularly preferably hydrogen;
R⁴ is hydrogen, C₁-C₆-alkyl, preferably C₁-C₃-alkyl, C₁-C₆-haloalkyl, preferably C₁-C₃-haloalkyl, and particularly preferably hydrogen;
R⁵ is hydrogen, C₁-C₄-alkyl or C₁-C₄-alkoxy;
R⁶ is hydrogen, C₁-C₄-alkyl, benzyl or C₁-C₄-alkylcarbonyl.

With a view to the use as fungicides, particularly preferred compounds of the formula I are the compounds of the formulae I.a, I.b and I.c in which R³ and R⁴ are each hydrogen, R² is hydroxyl, chlorine or methyl and (R^{a})ₙ is 2-methyl-4-chloro (compounds I.a.1, I.b.1 and I.c.1). Examples of these are compounds I.a.1, I.b.1 and I.c.1 in which R² and R¹ are each hydroxyl. Other examples are compounds I.a.1, I.b.1 and I.c.1 in which R² and R¹ are each chlorine. Other examples are compounds I.a.1, I.b.1 and I.c.1 in which R² is methyl and R¹ is chlorine. Other examples are compounds I.a.1, I.b.1 and I.c.1 in which R² is methyl and R¹ is chlorine. Other examples are compounds I.a.1, I.b.1 and I.c.1 in which R² is chlorine and R¹ is NR⁷R⁸, where R⁷, R⁸ together have in each case the meanings given in one row of Table A. Other examples are compounds I.a.1, I.b.1 and I.c.1 in which R² is methyl and R¹ is NR⁷R⁸, where R⁷, R⁸ together have in each case the meanings given in one row of Table A.

TABLE A

| No. | R⁷ | R⁸ |
|---|---|---|
| A-1 | CH₃ | H |
| A-2 | CH₃ | CH₃ |
| A-3 | CH₂CH₃ | H |
| A-4 | CH₂CH₃ | CH₃ |
| A-5 | CH₂CH₃ | CH₂CH₃ |
| A-6 | CH₂CF₃ | H |
| A-7 | CH₂CF₃ | CH₃ |
| A-8 | CH₂CF₃ | CH₂CH₃ |
| A-9 | CH₂CCl₃ | H |
| A-10 | CH₂CCl₃ | CH₃ |
| A-11 | CH₂CCl₃ | CH₂CH₃ |
| A-12 | CH₂CH₂CH₃ | H |
| A-13 | CH₂CH₂CH₃ | CH₃ |
| A-14 | CH₂CH₂CH₃ | CH₂CH₃ |
| A-15 | CH₂CH₂CH₃ | CH₂CH₂CH₃ |
| A-16 | CH(CH₃)₂ | H |
| A-17 | CH(CH₃)₂ | CH₃ |
| A-18 | CH(CH₃)₂ | CH₂CH₃ |
| A-19 | CH₂CH₂CH₂CH₃ | H |
| A-20 | CH₂CH₂CH₂CH₃ | CH₃ |
| A-21 | CH₂CH₂CH₂CH₃ | CH₂CH₃ |
| A-22 | CH₂CH₂CH₂CH₃ | CH₂CH₂CH₃ |
| A-23 | CH₂CH₂CH₂CH₃ | CH₂CH₂CH₂CH₃ |
| A-24 | (±)CH(CH₃)—CH₂CH₃ | H |
| A-25 | (±)CH(CH₃)—CH₂CH₃ | CH₃ |
| A-26 | (±)CH(CH₃)—CH₂CH₃ | CH₂CH₃ |
| A-27 | (S)CH(CH₃)—CH₂CH₃ | H |
| A-28 | (S)CH(CH₃)—CH₂CH₃ | CH₃ |
| A-29 | (S)CH(CH₃)—CH₂CH₃ | CH₂CH₃ |
| A-30 | (R)CH(CH₃)—CH₂CH₃ | H |
| A-31 | (R)CH(CH₃)—CH₂CH₃ | CH₃ |
| A-32 | (R)CH(CH₃)—CH₂CH₃ | CH₂CH₃ |
| A-33 | (±)CH(CH₃)—CH(CH₃)₂ | H |
| A-34 | (±)CH(CH₃)—CH(CH₃)₂ | CH₃ |
| A-35 | (±)CH(CH₃)—CH(CH₃)₂ | CH₂CH₃ |
| A-36 | (S)CH(CH₃)—CH(CH₃)₂ | H |
| A-37 | (S)CH(CH₃)—CH(CH₃)₂ | CH₃ |
| A-38 | (S)CH(CH₃)—CH(CH₃)₂ | CH₂CH₃ |
| A-39 | (R)CH(CH₃)—CH(CH₃)₂ | H |
| A-40 | (R)CH(CH₃)—CH(CH₃)₂ | CH₃ |
| A-41 | (R)CH(CH₃)—CH(CH₃)₂ | CH₂CH₃ |
| A-42 | (±)CH(CH₃)—C(CH₃)₃ | H |
| A-43 | (±)CH(CH₃)—C(CH₃)₃ | CH₃ |
| A-44 | (±)CH(CH₃)—C(CH₃)₃ | CH₂CH₃ |
| A-45 | (S)CH(CH₃)—C(CH₃)₃ | H |
| A-46 | (S)CH(CH₃)—C(CH₃)₃ | CH₃ |
| A-47 | (S)CH(CH₃)—C(CH₃)₃ | CH₂CH₃ |
| A-48 | (R)CH(CH₃)—C(CH₃)₃ | H |
| A-49 | (R)CH(CH₃)—C(CH₃)₃ | CH₃ |
| A-50 | (R)CH(CH₃)—C(CH₃)₃ | CH₂CH₃ |
| A-51 | (±)CH(CH₃)—CF₃ | H |
| A-52 | (±)CH(CH₃)—CF₃ | CH₃ |
| A-53 | (±)CH(CH₃)—CF₃ | CH₂CH₃ |
| A-54 | (S)CH(CH₃)—CF₃ | H |
| A-55 | (S)CH(CH₃)—CF₃ | CH₃ |
| A-56 | (S)CH(CH₃)—CF₃ | CH₂CH₃ |
| A-57 | (R)CH(CH₃)—CF₃ | H |
| A-58 | (R)CH(CH₃)—CF₃ | CH₃ |
| A-59 | (R)CH(CH₃)—CF₃ | CH₂CH₃ |
| A-60 | (±)CH(CH₃)—CCl₃ | H |
| A-61 | (±)CH(CH₃)—CCl₃ | CH₃ |
| A-62 | (±)CH(CH₃)—CCl₃ | CH₂CH₃ |
| A-63 | (S)CH(CH₃)—CCl₃ | H |
| A-64 | (S)CH(CH₃)—CCl₃ | CH₃ |
| A-65 | (S)CH(CH₃)—CCl₃ | CH₂CH₃ |
| A-66 | (R)CH(CH₃)—CCl₃ | H |
| A-67 | (R)CH(CH₃)—CCl₃ | CH₃ |
| A-68 | (R)CH(CH₃)—CCl₃ | CH₂CH₃ |
| A-69 | CH₂CF₂CF₃ | H |
| A-70 | CH₂CF₂CF₃ | CH₃ |
| A-71 | CH₂CF₂CF₃ | CH₂CH₃ |
| A-72 | CH₂(CF₂)₂CF₃ | H |
| A-73 | CH₂(CF₂)₂CF₃ | CH₃ |
| A-74 | CH₂(CF₂)₂CF₃ | CH₂CH₃ |
| A-75 | CH₂C(CH₃)═CH₂ | H |
| A-76 | CH₂C(CH₃)═CH₂ | CH₃ |
| A-77 | CH₂C(CH₃)═CH₂ | CH₂CH₃ |
| A-78 | CH₂CH═CH₂ | H |
| A-79 | CH₂CH═CH₂ | CH₃ |
| A-80 | CH₂CH═CH₂ | CH₂CH₃ |
| A-81 | CH(CH₃)CH═CH₂ | H |
| A-82 | CH(CH₃)CH═CH₂ | CH₃ |
| A-83 | CH(CH₃)CH═CH₂ | CH₂CH₃ |
| A-84 | CH(CH₃)C(CH₃)═CH₂ | H |
| A-85 | CH(CH₃)C(CH₃)═CH₂ | CH₃ |
| A-86 | CH(CH₃)C(CH₃)═CH₂ | CH₂CH₃ |
| A-87 | CH₂C≡CH | H |
| A-88 | CH₂C≡CH | CH₃ |
| A-89 | CH₂C≡CH | CH₂CH₃ |
| A-90 | cyclopentyl | H |
| A-91 | cyclopentyl | CH₃ |
| A-92 | cyclopentyl | CH₂CH₃ |
| A-93 | cyclohexyl | H |
| A-94 | cyclohexyl | CH₃ |
| A-95 | cyclohexyl | CH₂CH₃ |
| A-96 | CH₂—C₆H₅ | H |
| A-97 | CH₂—C₆H₅ | CH₃ |
| A-98 | CH₂—C₆H₅ | CH₂CH₃ |

| | |
|---|---|
| A-99 | —(CH₂)₂CH═CH—CH₂— |
| A-100 | —(CH₂)₂C(CH₃)═CHCH₂— |
| A-101 | —(CH₂)₂CH(CH₃)(CH₂)₂— |
| A-102 | —(CH₂)₃CHFCH₂— |
| A-103 | —(CH₂)₂CHF(CH₂)₂— |
| A-104 | —(CH₂)₂CHF(CH₂)₃— |
| A-105 | —(CH₂)₂CH(CF₃)(CH₂)₂— |
| A-106 | —(CH₂)₂O(CH₂)₂— |
| A-107 | —(CH₂)₂S(CH₂)₂— |
| A-108 | —(CH₂)₅— |
| A-109 | —(CH₂)₄— |
| A-110 | —CH₂CH═CHCH₂— |
| A-111 | —CH(CH₃)(CH₂)₃— |
| A-112 | —CH₂CH(CH₃)(CH₂)₂— |
| A-113 | —CH(CH₃)—(CH₂)₂—CH(CH₃)— |
| A-114 | —CH(CH₃)—(CH₂)₄— |
| A-115 | —CH₂—CH(CH₃)—(CH₂)₃— |
| A-116 | —(CH₂)—CH(CH₃)—CH₂—CH(CH₃)—CH₂— |
| A-117 | —CH(CH₂CH₃)—(CH₂)₄— |
| A-118 | —(CH₂)₂—CHOH—(CH₂)₂— |
| A-119 | —(CH₂)—CH═CH—(CH₂)₂— |
| A-120 | —(CH₂)₆— |
| A-121 | —CH(CH₃)—(CH₂)₅— |
| A-122 | —(CH₂)₂—N(CH₃)—(CH₂)₂— |
| A-123 | —N═CH—CH═CH— |
| A-124 | —N═C(CH₃)—CH═C(CH₃)— |
| A-125 | —N═C(CF₃)—CH═C(CF₃)— |

With a view to the use as fungicide, particularly preferred compounds of the formula I are furthermore the compounds of the formulae I.a, I.b and I.c in which R³ and R⁴ are each hydrogen, R² is hydroxyl, chlorine or methyl and (R^{a})ₙ is 2-fluoro-4-methyl (compounds I.a.2, I.b.2 and I.c.2). Examples of these are the compounds I.a.2, I.b.2 and I.c.2 in which R² and R¹ are each hydroxyl. Other examples are the compounds I.a.2, I.b.2 and I.c.2 in which R² and R¹ are each chlorine. Other examples are the compounds I.a.2, I.b.2 and I.c.2 in which R² is methyl and R¹ is chlorine. Other examples are the compounds I.a.2, I.b.2 and I.c.2 in which R² is chlorine and R¹ is NR⁷R⁸, where R⁷, R⁸ together have in each case the meanings given in one row of Table A. Other examples are the compounds I.a.2, I.b.2 and I.c.2 in which R² is methyl and R¹ is NR⁷R⁸, where R⁷, R⁸ together have in each case the meanings given in one row of Table A.

With a view to the use as fungicide, particularly preferred compounds of the formula I are furthermore the compounds of the formulae I.a, I.b and I.c in which R³ and R⁴ are each hydrogen, R² is hydroxyl, chlorine or methyl and (R^{a})ₙ is 2,6-dimethyl (compounds I.a.3, I.b.3 and I.c.3). Examples of these are the compounds I.a.3, I.b.3 and I.c.3 in which R² and R¹ are each hydroxyl. Other examples are the compounds I.a.3, I.b.3 and I.c.3 in which R² and R¹ are each chlorine. Other examples are the compounds I.a.3, I.b.3 and I.c.3 in which R² is methyl and R¹ is chlorine. Other examples are the compounds I.a.3, I.b.3 and I.c.3 in which R² is chlorine and R¹ is NR⁷R⁸, where R⁷, R⁸ together have in each case the meanings given in one row of Table A. Other examples are the compounds I.a.3, I.b.3 and I.c.3 in which R² is methyl and R¹ is NR⁷R⁸, where R⁷, R⁸ together have in each case the meanings given in one row of Table A.

With a view to the use as fungicide, particularly preferred compounds of the formula I are furthermore the compounds of the formulae I.a, I.b and I.c in which R³ and R⁴ are each hydrogen, R² is hydroxyl, chlorine or methyl and (R^{a})ₙ is 2,4,6-trimethyl (compounds I.a.4, I.b.4 and I.c.4). Examples of these are the compounds I.a.4, I.b.4 and I.c.4 in which R² and R¹ are each hydroxyl. Other examples are the compounds I.a.4, I.b.4 and I.c.4 in which R² and R¹ are each chlorine. Other examples are the compounds I.a.4, I.b.4 and I.c.4 in which R² is methyl and R¹ is chlorine. Other examples are the compounds I.a.4, I.b.4 and I.c.4 in which R² is chlorine and R¹ is NR⁷R⁸, where R⁷, R⁸ together have in each case the meanings given in one row of Table A. Other examples are the compounds I.a.4, I.b.4 and I.c.4 in which R² is methyl and R¹ is NR⁷R⁸, where R⁷, R⁸ together have in each case the meanings given in one row of Table A.

With a view to the use as fungicide, particularly preferred compounds of the formula I are furthermore the compounds of the formulae I.a, I.b and I.c in which R³ and R⁴ are each hydrogen, R² is hydroxyl, chlorine or methyl and (R^{a})ₙ is 2,6-difluoro-4-methyl (compounds I.a.5, I.b.5 and I.c.5). Examples of these are the compounds I.a.5, I.b.5 and I.c.5 in which R² and R¹ are each hydroxyl. Other examples are the compounds I.a.5, I.b.5 and I.c.5 in which R² and R¹ are each chlorine. Other examples are the compounds I.a.5, I.b.5 and I.c.5 in which R² is methyl and R¹ is chlorine. Other examples are the compounds I.a.5, I.b.5 and I.c.5 in which R² is chlorine and R¹ is NR⁷R⁸, where R⁷, R⁸ together have in each case the meanings given in one row of Table A. Other examples are the compounds I.a.5, I.b.5 and I.c.5 in which R² is methyl and R¹ is NR⁷R⁸, where R⁷, R⁸ together have in each case the meanings given in one row of Table A.

With a view to the use as fungicide, particularly preferred compounds of the formula I are furthermore the compounds of the formulae I.a, I.b and I.c in which R³ and R⁴ are each hydrogen, R² is hydroxyl, chlorine or methyl and (R^{a})ₙ is 2,6-difluoro-4-cyano (compounds I.a.6, I.b.6 and I.c.6). Examples of these are the compounds I.a.6, I.b.6 and I.c.6 in which R² and R¹ are each hydroxyl. Other examples are the compounds I.a.6, I.b.6 and I.c.6 in which R² and R¹ are each chlorine. Other examples are the compounds I.a.6, I.b.6 and I.c.6 in which R² is methyl and R¹ is chlorine. Other examples are the compounds I.a.6, I.b.6 and I.c.6 in which R² is chlorine and R¹ is NR⁷R⁸, where R⁷, R⁸ together have in each case the meanings given in one row of Table A. Other examples are the compounds I.a.6, I.b.6 and I.c.6 in which R² is methyl and R¹ is NR⁷R⁸, where R⁷, R⁸ together have in each case the meanings given in one row of Table A.

With a view to the use as fungicide, particularly preferred compounds of the formula I are furthermore the compounds of the formulae I.a, I.b and I.c in which R³ and R⁴ are each hydrogen, R² is hydroxyl, chlorine or methyl and (R^{a})ₙ is 2,6-difluoro-4-methoxycarbonyl (compounds I.a.7, I.b.7 and I.c.7). Examples of these are the compounds I.a.7, I.b.7 and I.c.7 in which R² and R¹ are each hydroxyl. Other examples are the compounds I.a.7, I.b.7 and I.c.7 in which R² and R¹ are each chlorine. Other examples are the compounds I.a.7, I.b.7 and I.c.7 in which R² is methyl and R¹ is chlorine. Other examples are the compounds I.a.7, I.b.7 and I.c.7 in which R² is chlorine and R¹ is NR⁷R⁸, where R⁷, R⁸ together have in each case the meanings given in one row of Table A. Other examples are the compounds I.a.7, I.b.7 and I.c.7 in which R² is methyl and R¹ is NR⁷R⁸, where R⁷, R⁸ together have in each case the meanings given in one row of Table A.

With a view to the use as fungicide, particularly preferred compounds of the formula I are furthermore the compounds of the formulae I.a, I.b and I.c in which R³ and R⁴ are each hydrogen, R² is hydroxyl, chlorine or methyl and (R^{a})ₙ is 2-trifluoromethyl-4-fluoro (compounds I.a.8, I.b.8 and I.c.8). Examples of these are the compounds I.a.8, I.b.8 and I.c.8 in which R² and R¹ are each hydroxyl. Other examples are the compounds I.a.8, I.b.8 and I.c.8 in which R² and R¹ are each chlorine. Other examples are the compounds I.a.8, I.b.8 and I.c.8 in which R² is methyl and R¹ is chlorine. Other examples are the compounds I.a.8, I.b.8 and I.c.8 in which R² is chlorine and R¹ is NR⁷R⁸, where R⁷, R⁸ together have in each case the meanings given in one row of Table A. Other examples are the compounds I.a.8, I.b.8 and I.c.8 in which R² is methyl and R¹ is NR⁷R⁸, where R⁷, R⁸ together have in each case the meanings given in one row of Table A.

With a view to the use as fungicide, particularly preferred compounds of the formula I are furthermore the compounds of the formulae I.a, I.b and I.c in which R³ and R⁴ are each hydrogen, R² is hydroxyl, chlorine or methyl and (R^{a})ₙ is 2-trifluoromethyl-5-fluoro (compounds I.a.9, I.b.9 and I.c.9). Examples of these are the compounds I.a.9, I.b.9 and I.c.9 in which R² and R¹ are each hydroxyl. Other examples are the compounds I.a.9, I.b.9 and I.c.9 in which R² and R¹ are each chlorine. Other examples are the compounds I.a.9, I.b.9 and I.c.9 in which R² is methyl and R¹ is chlorine. Other examples are the compounds I.a.9, I.b.9 and I.c.9 in which R² is chlorine and R¹ is NR⁷R⁸, where R⁷, R⁸ together have in each case the meanings given in one row of Table A. Other examples are the compounds I.a.9, I.b.9 and I.c.9 in which R² is methyl and R¹ is NR⁷R⁸, where R⁷, R⁸ together have in each case the meanings given in one row of Table A.

With a view to the use as fungicide, particularly preferred compounds of the formula I are furthermore the compounds of the formulae I.a, I.b and I.c in which R³ and R⁴ are each hydrogen, R² is hydroxyl, chlorine or methyl and (R^{a})ₙ is 2-trifluoromethyl-5-chloro (compounds I.a.10, I.b.10 and I.c.10). Examples of these are the compounds I.a.10, I.b.10 and I.c.10 in which R² and R¹ are each hydroxyl. Other examples are the compounds I.a.10, I.b.10 and I.c.10 in which R² and R¹ are each chlorine. Other examples are the compounds I.a.10, I.b.10 and I.c.10 in which R² is methyl and R¹ is chlorine. Other examples are the compounds I.a.10, I.b.10 and I.c.10 in which R² is chlorine and R¹ is NR⁷R⁸, where R⁷, R⁸ together have in each case the meanings given in one row of Table A. Other examples are the compounds I.a.10, I.b.10 and I.c.10 in which R² is methyl and R¹ is NR⁷R⁸, where R⁷, R⁸ together have in each case the meanings given in one row of Table A.

With a view to the use as fungicide, particularly preferred compounds of the formula I are furthermore the compounds of the formulae I.a, I.b and I.c in which R³ and R⁴ are each hydrogen, R² is hydroxyl, chlorine or methyl and (R^{a})ₙ is 2-chloro-6-fluoro (compounds I.a.11, I.b.11 and I.c.11). Examples of these are the compounds I.a.11, I.b.11 and I.c.11 in which R² and R¹ are each hydroxyl. Other examples are the compounds I.a.11, I.b.11 and I.c.11 in which R² and R¹ are each chlorine. Other examples are the compounds I.a.11, I.b.11 and I.c.11 in which R² is methyl and R¹ is chlorine. Other examples are the compounds I.a.11, I.b.11 and I.c.11 in which R² is chlorine and R¹ is NR⁷R⁸, where R⁷, R⁸ together have in each case the meanings given in one row of Table A. Other examples are the compounds I.a.11, I.b.11 and I.c.11 in which R² is methyl and R¹ is NR⁷R⁸, where R⁷, R⁸ together have in each case the meanings given in one row of Table A.

With a view to the use as fungicide, particularly preferred compounds of the formula I are furthermore the compounds of the formulae I.a, I.b and I.c in which R³ and R⁴ are each hydrogen, R² is hydroxyl, chlorine or methyl and (R^{a})ₙ is 2,6-difluoro (compounds I.a.12, I.b.12 and I.c.12). Examples of these are the compounds I.a.12, I.b.12 and I.c.12 in which R² and R¹ are each hydroxyl. Other examples are the compounds I.a.12, I.b.12 and I.c.12 in which R² and R¹ are each chlorine. Other examples are the compounds I.a.12, I.b.12 and I.c.12 in which R² is methyl and R¹ is chlorine. Other examples are the compounds I.a.12, I.b.12 and I.c.12 in which R² is chlorine and R¹ is NR⁷R⁸, where R⁷, R⁸ together have in each case the meanings given in one row of Table A. Other examples are the compounds I.a.12, I.b.12 and I.c.12 in which R² is methyl and R¹ is NR⁷R⁸, where R⁷, R⁸ together have in each case the meanings given in one row of Table A.

With a view to the use as fungicide, particularly preferred compounds of the formula I are furthermore the compounds of the formulae I.a, I.b and I.c in which R³ and R⁴ are each hydrogen, R² is hydroxyl, chlorine or methyl and (R^{a})ₙ is 2,6-dichloro (compounds I.a.13, I.b.13 and I.c.13). Examples of these are the compounds I.a.13, I.b.13 and I.c.13 in which R² and R¹ are each hydroxyl. Other examples are the compounds I.a.13, I.b.13 and I.c.13 in which R² and R¹ are each chlorine. Other examples are the compounds I.a.13, I.b.13 and I.c.13 in which R² is methyl and R¹ is chlorine. Other examples are the compounds I.a.13, I.b.13 and I.c.13 in which R² is chlorine and R¹ is NR⁷R⁸, where R⁷, R⁸ together have in each case the meanings given in one row of Table A. Other examples are the compounds I.a.13, I.b.13 and I.c.13 in which R² is methyl and R¹ is NR⁷R⁸, where R⁷, R⁸ together have in each case the meanings given in one row of Table A.

With a view to the use as fungicide, particularly preferred compounds of the formula I are furthermore the compounds of the formulae I.a, I.b and I.c in which R³ and R⁴ are each hydrogen, R² is hydroxyl, chlorine or methyl and (R^{a})ₙ is 2-fluoro-6-methyl (compounds I.a.14, I.b.14 and I.c.14). Examples of these are the compounds I.a.14, I.b.14 and I.c.14 in which R² and R¹ are each hydroxyl. Other examples are the compounds I.a.14, I.b.14 and I.c.14 in which R² and R¹ are each chlorine. Other examples are the compounds I.a.14, I.b.14 and I.c.14 in which R² is methyl and R¹ is chlorine. Other examples are the compounds I.a.14, I.b.14 and I.c.14 in which R² is chlorine and R¹ is NR⁷R³, where R⁷, R⁸ together have in each case the meanings given in one row of Table A. Other examples are the compounds I.a.14, I.b.14 and I.c.14 in which R² is methyl and R¹ is NR⁷R⁸, where R⁷, R⁸ together have in each case the meanings given in one row of Table A.

With a view to the use as fungicide, particularly preferred compounds of the formula I are furthermore the compounds of the formulae I.a, I.b and I.c in which R³ and R⁴ are each hydrogen, R² is hydroxyl, chlorine or methyl and (R^{a})ₙ is 2,4,6-trifluoro (compounds I.a.15, I.b.15 and I.c.15). Examples of these are the compounds I.a.15, I.b.15 and I.c.15 in which R² and R¹ are each hydroxyl. Other examples are the compounds I.a.15, I.b.15 and I.c.15 in which R² and R¹ are each chlorine. Other examples are the compounds I.a.15, I.b.15 and I.c.15 in which R² is methyl and R¹ is chlorine. Other examples are the compounds I.a.15, I.b.15 and I.c.15 in which R² is chlorine and R¹ is NR⁷R⁸, where R⁷, R⁸ together have in each case the meanings given in one row of Table A. Other examples are the compounds I.a.15, I.b.15 and I.c.15 in which R² is methyl and R¹ is NR⁷R⁸, where R⁷, R⁸ together have in each case the meanings given in one row of Table A.

With a view to the use as fungicide, particularly preferred compounds of the formula I are furthermore the compounds of the formulae I.a, I.b and I.c in which R³ and R⁴ are each hydrogen, R² is hydroxyl, chlorine or methyl and (R^{a})ₙ is 2,6-difluoro-4-methoxy (compounds I.a.16, I.b.16 and I.c.16). Examples of these are the compounds I.a.16, I.b.16 and I.c.16 in which R² and R¹ are each hydroxyl. Other examples are the compounds I.a.16, I.b.16 and I.c.16 in which R² and R¹ are each chlorine. Other examples are the compounds I.a.16, I.b.16 and I.c.16 in which R² is methyl and R¹ is chlorine. Other examples are the compounds I.a.16, I.b.16 and I.c.16 in which R² is chlorine and R¹ is NR⁷R⁸, where R⁷, R⁸ together have in each case the meanings given in one row of Table A. Other examples are the compounds I.a.16, I.b.16 and I.c.16 in which R² is methyl and R¹ is NR⁷R⁸, where R⁷, R⁸ together have in each case the meanings given in one row of Table A.

With a view to the use as fungicide, particularly preferred compounds of the formula I are furthermore the compounds of the formulae I.a, I.b and I.c in which R³ and R⁴ are each hydrogen, R² is hydroxyl, chlorine or methyl and (R^{a})ₙ is 2,3,4,5,6-pentafluoro (compounds I.a.17, I.b.17 and I.c.17). Examples of these are the compounds I.a.17, I.b.17 and I.c.17 in which R² and R¹ are each hydroxyl. Other examples are the compounds I.a.17, I.b.17 and I.c.17 in which R² and R¹ are each chlorine. Other examples are the compounds I.a.17, I.b.17 and I.c.17 in which R² is methyl and R¹ is chlorine. Other examples are the compounds I.a.17, I.b.17 and I.c.17 in which R² is chlorine and R¹ is NR⁷R⁸, where R⁷, R³ together have in each case the meanings given in one row of Table A. Other examples are the compounds I.a.17, I.b.17 and I.c.17 in which R² is methyl and R¹ is NR⁷R⁸, where R⁷, R⁸ together have in each case the meanings given in one row of Table A.

With a view to the use as fungicide, particularly preferred compounds of the formula I are furthermore the compounds of the formulae I.a, I.b and I.c in which R³ and R⁴ are each hydrogen, R² is hydroxyl, chlorine or methyl and (R^{a})ₙ is 2-methyl-4-fluoro (compounds I.a.18, I.b.18 and I.c.18). Examples of these are the compounds I.a.18, I.b.18 and I.c.18 in which R² and R¹ are each hydroxyl. Other examples are the compounds I.a.18, I.b.18 and I.c.18 in which R² and R¹ are each chlorine. Other examples are the compounds I.a.18, I.b.18 and I.c.18 in which R² is methyl and R¹ is chlorine. Other examples are the compounds I.a.18, I.b.18 and I.c.18 in which R² is chlorine and R¹ is NR⁷R⁸, where R⁷, R⁸ together have in each case the meanings given in one row of Table A. Other examples are the compounds I.a.18, I.b.18 and I.c.18 in which R² is methyl and R¹ is NR⁷R⁸, where R⁷, R⁸ together have in each case the meanings given in one row of Table A.

With a view to the use as fungicide, particularly preferred compounds of the formula I are furthermore the compounds of the formulae I.a, I.b and I.c in which R³ and R⁴ are each hydrogen, R² is hydroxyl, chlorine or methyl and (R^{a})ₙ is 2-fluoro-6-methoxy (compounds I.a.19, I.b.19 and I.c.19). Examples of these are the compounds I.a.19, I.b.19 and I.c.19 in which R² and R¹ are each hydroxyl. Other examples are the compounds I.a.19, I.b.19 and I.c.19 in which R² and R¹ are each chlorine. Other examples are the compounds I.a.19, I.b.19 and I.c.19 in which R² is methyl and R¹ is chlorine. Other examples are the compounds I.a.19, I.b.19 and I.c.19 in which R² is chlorine and R¹ is NR⁷R⁸, where R⁷, R⁸ together have in each case the meanings given in one row of Table A. Other examples are the compounds I.a.19, I.b.19 and I.c.19 in which R² is methyl and R¹ is NR⁷R⁸, where R⁷, R⁸ together have in each case the meanings given in one row of Table A.

With a view to the use as fungicide, particularly preferred compounds of the formula I are furthermore the compounds of the formulae I.a, I.b and I.c in which R³ and R⁴ are each hydrogen, R² is hydroxyl, chlorine or methyl and (R^{a})ₙ is 2,4-difluoro (compounds I.a.20, I.b.20 and I.c.20). Examples of these are the compounds I.a.20, I.b.20 and I.c.20 in which R² and R¹ are each hydroxyl. Other examples are the compounds I.a.20, I.b.20 and I.c.20 in which R² and R¹ are each chlorine. Other examples are the compounds I.a.20, I.b.20 and I.c.20 in which R² is methyl and R¹ is chlorine. Other examples are the compounds I.a.20, I.b.20 and I.c.20 in which R² is chlorine and R¹ is NR⁷R⁸, where R⁷, R⁸ together have in each case the meanings given in one row of Table A. Other examples are the compounds I.a.20, I.b.20 and I.c.20 in which R² is methyl and R¹ is NR⁷R³, where R⁷, R⁸ together have in each case the meanings given in one row of Table A.

With a view to the use as fungicide, particularly preferred compounds of the formula I are furthermore the compounds of the formulae I.a, I.b and I.c in which R³ and R⁴ are each hydrogen, R² is hydroxyl, chlorine or methyl and (R^{a})ₙ is 2-fluoro-4-chloro (compounds I.a.21, I.b.21 and I.c.21). Examples of these are the compounds I.a.21, I.b.21 and I.c.21 in which R² and R¹ are each hydroxyl. Other examples are the compounds I.a.21, I.b.21 and I.c.21 in which R² and R¹ are each chlorine. Other examples are the compounds I.a.21, I.b.21 and I.c.21 in which R² is methyl and R¹ is chlorine. Other examples are the compounds I.a.21, I.b.21 and I.c.21 in which R² is chlorine and R¹ is NR⁷R⁸, where R⁷, R⁸ together have in each case the meanings given in one row of Table A. Other examples are the compounds I.a.21, I.b.21 and I.c.21 in which R² is methyl and R¹ is NR⁷R⁸, where R⁷, R⁸ together have in each case the meanings given in one row of Table A.

With a view to the use as fungicide, particularly preferred compounds of the formula I are furthermore the compounds of the formulae I.a, I.b and I.c in which R³ and R⁴ are each hydrogen, R² is hydroxyl, chlorine or methyl and (R^{a})ₙ is 2-chloro-4-fluoro (compounds I.a.22, I.b.22 and I.c.22). Examples of these are the compounds I.a.22, I.b.22 and I.c.22 in which R² and R¹ are each hydroxyl. Other examples are the compounds I.a.22, I.b.22 and I.c.22 in which R² and R¹ are each chlorine. Other examples are the compounds I.a.22, I.b.22 and I.c.22 in which R² is methyl and R¹ is chlorine. Other examples are the compounds I.a.22, I.b.22 and I.c.22 in which R² is chlorine and R¹ is NR⁷R⁸, where R⁷, R⁸ together have in each case the meanings given in one row of Table A. Other examples are the compounds I.a.22, I.b.22 and I.c.22 in which R² is methyl and R¹ is NR⁷R⁸, where R⁷, R⁸ together have in each case the meanings given in one row of Table A.

With a view to the use as fungicide, particularly preferred compounds of the formula I are furthermore the compounds of the formulae I.a, I.b and I.c in which R³ and R⁴ are each hydrogen, R² is hydroxyl, chlorine or methyl and (R^{a})ₙ is 2,3-difluoro (compounds I.a.23, I.b.23 and I.c.23). Examples of these are the compounds I.a.23, I.b.23 and I.c.23 in which R² and R¹ are each hydroxyl. Other examples are the compounds I.a.23, I.b.23 and I.c.23 in which R² and R¹ are each chlorine. Other examples are the compounds I.a.23, I.b.23 and I.c.23 in which R² is methyl and R¹ is chlorine. Other examples are the compounds I.a.23, I.b.23 and I.c.23 in which R² is chlorine and R¹ is NR⁷R⁸, where R⁷, R⁸ together have in each case the meanings given in one row of Table A. Other examples are the compounds I.a.23, I.b.23 and I.c.23 in which R² is methyl and R¹ is NR⁷R⁸, where R⁷, R⁸ together have in each case the meanings given in one row of Table A.

With a view to the use as fungicide, particularly preferred compounds of the formula I are furthermore the compounds of the formulae I.a, I.b and I.c in which R³ and R⁴ are each hydrogen, R² is hydroxyl, chlorine or methyl and (R^{a})ₙ is 2,5-difluoro (compounds I.a.24, I.b.24 and I.c.24). Examples of these are the compounds I.a.24, I.b.24 and I.c.24 in which R² and R¹ are each hydroxyl. Other examples are the compounds I.a.24, I.b.24 and I.c.24 in which R² and R¹ are each chlorine. Other examples are the compounds I.a.24, I.b.24 and I.c.24 in which R² is methyl and R¹ is chlorine. Other examples are the compounds I.a.24, I.b.24 and I.c.24 in which R² is chlorine and R¹ is NR⁷R⁸, where R⁷, R⁸ together have in each case the meanings given in one row of Table A. Other examples are the compounds I.a.24, I.b.24 and I.c.24 in which R² is methyl and R¹ is NR⁷R⁸, where R⁷, R⁸ together have in each case the meanings given in one row of Table A.

With a view to the use as fungicide, particularly preferred compounds of the formula I are furthermore the compounds of the formulae I.a, I.b and I.c in which R³ and R⁴ are each hydrogen, R² is hydroxyl, chlorine or methyl and (R^{a})ₙ is 2,3,4-trifluoro (compounds I.a.25, I.b.25 and I.c.25). Examples of these are the compounds I.a.25, I.b.25 and I.c.25 in which R² and R¹ are each hydroxyl. Other examples are the compounds I.a.25, I.b.25 and I.c.25 in which R² and R¹ are each chlorine. Other examples are the compounds I.a.25, I.b.25 and I.c.25 in which R² is methyl and R¹ is chlorine. Other examples are the compounds I.a.25, I.b.25 and I.c.25 in which R² is chlorine and R¹ is NR⁷R⁸, where R⁷, R⁸ together have in each case the meanings given in one row of Table A. Other examples are the compounds I.a.25, I.b.25 and I.c.25 in which R² is methyl and R¹ is NR⁷R⁸, where R⁷, R⁸ together have in each case the meanings given in one row of Table A.

With a view to the use as fungicide, particularly preferred compounds of the formula I are furthermore the compounds of the formulae I.a, I.b and I.c in which R³ and R⁴ are each hydrogen, R² is hydroxyl, chlorine or methyl and (R^{a})ₙ is 2,4-dimethyl (compounds I.a.26, I.b.26 and I.c.26). Examples of these are the compounds I.a.26, I.b.26 and I.c.26 in which R² and R¹ are each hydroxyl. Other examples are the compounds I.a.26, I.b.26 and I.c.26 in which R² and R¹ are each chlorine. Other examples are the compounds I.a.26, I.b.26 and I.c.26 in which R² is methyl and R¹ is chlorine. Other examples are the compounds I.a.26, I.b.26 and I.c.26 in which R² is chlorine and R¹ is NR⁷R⁸, where R⁷, R⁸ together have in each case the meanings given in one row of Table A. Other examples are the compounds I.a.26, I.b.26 and I.c.26 in which R² is methyl and R¹ is NR⁷R⁸, where R⁷, R⁸ together have in each case the meanings given in one row of Table A.

The compounds of the formula I according to the invention can be prepared analogously to processes known per se of the prior art, according to the syntheses shown in the schemes below:

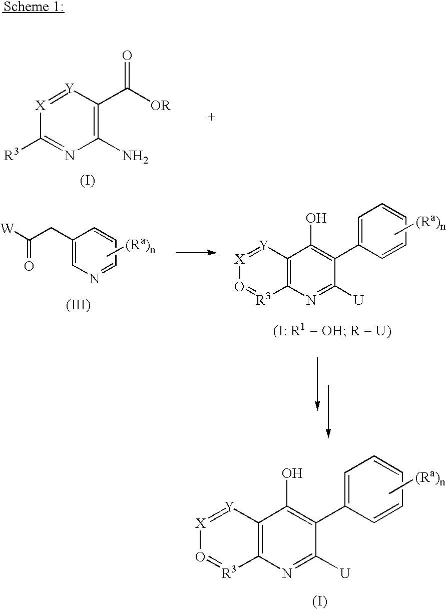

In scheme 1, n, R^{a}, R¹, R², R³, X and Y are as defined above. R is C₁-C₄-alkyl, in particular methyl or ethyl, W is C₁-C₆-alkyl, C₁-C₆-alkoxy, in particular methoxy or ethoxy, C₁-C₆-haloalkyl, optionally substituted C₃-C₈-cycloalkyl, optionally substituted C₅-C₈-cycloalkenyl, C₂-C₆-alkenyl or C₂-C₆-alkynyl and U is OH, C₁-C₆-alkyl, C₁-C₆-haloalkyl, optionally substituted C₃-C₈-cycloalkyl, optionally substituted C₅-C₈-cycloalkenyl, C₂-C₆-alkenyl or C₂-C₆-alkynyl.

According to scheme 1, in a first step, a hetarylamine of the formula II is condensed with a CH-acidic compound of the formula III. Examples of suitable CH-acidic compounds of the formula III are substituted (C₁-C₄)-alkyl phenylacetates and substituted benzyl (halo)alkyl ketones, benzyl cycloalkyl ketones, benzyl alkenyl ketones, benzyl cycloalkenyl ketones and benzyl alkynyl ketones. Examples of suitable hetarylamines of the formula II are 2-aminopyridine-3-carboxylic esters (2-aminonicotinic esters), 3-aminopyrazine-2-carboxylic esters and 4-aminopyrimidine-5-carboxylic esters.

Thus, when using 2-aminopyridine-3-carboxylic esters (2-aminonicotinic esters) are used, the compounds I.a where R¹=OH are obtained; when using 3-aminopyrazine-2-carboxylic esters, the compounds I.b where R¹=OH are obtained and when using 4-aminopyrimidine-5-carboxylic esters, the compounds I.c where R¹=OH are obtained.

The condensation is generally carried out in the presence of a Brønsted or Lewis acid as acidic catalyst or in the presence of a basic catalyst; see, for example, Organikum, 15th edition, VEB Deutscher Verlag der Wissenschaften, Berlin 1976, 552ff. Examples of suitable acidic catalysts are zinc chloride, phosphoric acid, hydrochloric acid, acetic acid and also mixtures of acetic acid and zinc chloride. Examples of suitable basic catalysts are inorganic and organic basic catalysts. Suitable inorganic basic catalysts are, for example, alkali metal or alkaline earth metal hydrides, preferably alkali metal hydrides, such as sodium hydride or potassium hydride, and alkali metal and alkaline earth metal hydroxides, such as sodium hydroxide or potassium hydroxide. When substituted phenylacetic esters are used, the condensation can furthermore be carried out in the presence of metallic sodium. Examples of suitable organic basic catalysts are alkali metal or alkaline earth metal alkoxides, such as sodium methoxide, sodium ethoxide, sodium n-propoxide, sodium isopropoxide, sodium n-butoxide, sodium sec-butoxide, sodium tert-butoxide, potassium methoxide, potassium ethoxide, potassium n-propoxide, potassium isopropoxide, potassium n-butoxide, potassium sec-butoxide, potassium tert-butoxide, secondary amines, such as ethyldiisopropylamine, and amidine bases, such as 1,5-diazabicyclo[4.3.0]non-5-ene (DBN) or 1,8-diazabicyclo[5.4.0]undec-7-ene (DBU).

The reaction can be carried out in the absence of a solvent or in a solvent. In the absence of a solvent, the CH-acidic compound III is usually employed in excess, based on the hetarylamine.

Condensation reactions of a hetarylamine of the formula II with a suitably substituted phenylacetic ester of the formula III are known in principle from the literature, for example from Archiv der Pharmazie, 290, 1957, 136, Chem. Ber. 96, 1963, 1868, Chem. Ber. 111, 1978, 2813-2824 or J. Heterocyclic Chem. 30, 909 (1963), and can be utilized in an analogous manner for preparing the compounds according to the invention.

In the condensation shown in scheme 1, when using phenylacetic esters of the formula II, compounds I are obtained in which R¹ and R² are each hydroxyl. If ketones of the formula III are employed, compounds I are obtained where R¹=hydroxyl and R²=C₁-C₆-(halo)alkyl, C₂-C₆-alkenyl, C₂-C₆-alkynyl, optionally substituted C₃-C₈-cycloalkyl or optionally substituted C₅-C₈-cycloalkenyl.

Such compounds I (R¹=OH) are of particular interest as intermediates for the preparation of other compounds I. In one or more steps, the OH group(s) in these compounds can be converted into other functional groups. To this end, the OH group(s) will generally initially be converted into halogen, in particular chlorine (see scheme 1a).

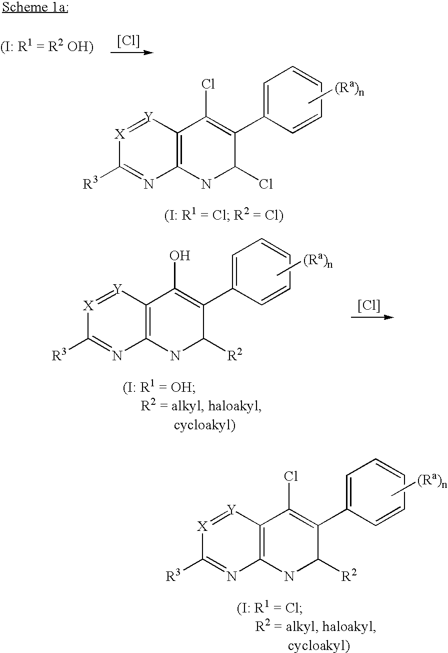

In scheme 1a, the variables n, R^{a}, R³, X and Y are as defined above. This conversion succeeds, for example, by reacting I {R¹=OH, R²=OH, C₁-C₆-(halo)alkyl, optionally substituted C₃-C₈-cycloalkyl} with a suitable halogenating agent (shown in scheme 1a for a chlorinating agent [Cl]). This method is known in principle, for example, from Archiv der Pharmazie, 290, 1957, p. 136 or J. Heterocyclic Chem., 30, 909 (1993).

Suitable halogenating agents are, for example, phosphorus trihalides, phosphorus oxyhalides or phosphorus pentahalides, such as phosphorus tribromide, phosphorus oxytribromide, and in particular chlorinating agents such as POCl₃, PCl₃/Cl₂ or PCl₅, and mixtures of these reagents. For the chlorination, preference is given to using a mixture of phosphorus pentachloride and phosphorus oxychloride. The reaction can be carried out in excess halogenating agent (POCl₃) or an inert solvent, such as, for example, acetonitrile or 1,2-dichloroethane.

This reaction is usually carried out between 10 and 180° C. For practical reasons, the reaction temperature frequently corresponds to the boiling point of the chlorinating agent (POCl₃) used or of the solvent. The process is, if appropriate, carried out with addition of N,N-dimethylformamide or nitrogen bases, such as, for example, N,N-dimethylaniline, in catalytic or stoichiometric amounts.

The monohalo compounds I obtained in this reaction, for example the chloro compound I {R¹=Cl; R²=C₁-C₆-(halo)alkyl, optionally substituted C₃-C₈-cycloalkyl} or the dichloro compound I {R¹=R²=Cl} can then be converted analogously to known processes from the prior art into other compounds I.

Compounds of the formula I in which R¹ is OR⁶ are obtained from the corresponding chloro compounds of the formula I {R¹=Cl, R²=alkyl, haloalkyl, cycloalkyl} by reaction with alkali metal hydroxides {OR⁶=OH}, alkali metal or alkaline earth metal alkoxides {OR⁶=O-alkyl, O-haloalkyl}[cf.: Heterocycles, Vol. 32, pp. 1327-1340 (1991); J. Heterocycl. Chem. Vol. 19, pp. 1565-1567 (1982); Geterotsikl. Soedin, pp. 400-402 (1991)]. The esterification of compounds where R¹=OH by methods known per se affords compounds I in which R¹ is O—C(O)R⁹. Compounds where R¹=OH can also be converted by etherification methods known per se into the corresponding compounds I in which R¹ is O-alkyl, O-haloalkyl or O-alkenyl.

Compounds of the formula I in which R¹ is cyano can be obtained from the corresponding chloro compounds of the formula I {R¹=Cl, R²=alkyl, haloalkyl, cycloalkyl} by reaction with alkali metal, alkaline earth metal or metal cyanides, such as NaCN, KCN or Zn(CN)₂ [cf.: Heterocycles, Vol. 39, pp. 345-356 (1994); Collect. Czech. Chem. Commun. Vol. 60, pp. 1386-1389 (1995); Acta Chim. Scand., Vol. 50, pp. 58-63 (1996)].

Compounds of the formula I in which R¹ is halogen and compounds of the formula I in which R¹ and R² are both halogen are therefore of particular interest as intermediates for preparing other compounds I. An overview over further such conversions is given by schemes 1b, 1c and 1d.

Thus, for example, as shown in scheme 1b, the monochloro compound I {R¹=Cl, R²=C₁-C₆-(halo)alkyl, optionally substituted C₃-C₈-cycloalkyl} can be reacted with an amine HNR⁷R⁸, in which R⁷ and R⁸ are as defined above, which gives a compound I in which R¹ is NR⁷R⁸ and R² is C₁-C₆-(halo)alkyl, optionally substituted C₃-C₈-cycloalkyl. If the dichloro compound I {R¹=R²=Cl} is reacted with an amine HNR⁷R⁸ in which R⁷ and R⁸ are as defined above, a compound I in which R¹ is chlorine and R² is NR⁷R⁸ is obtained.

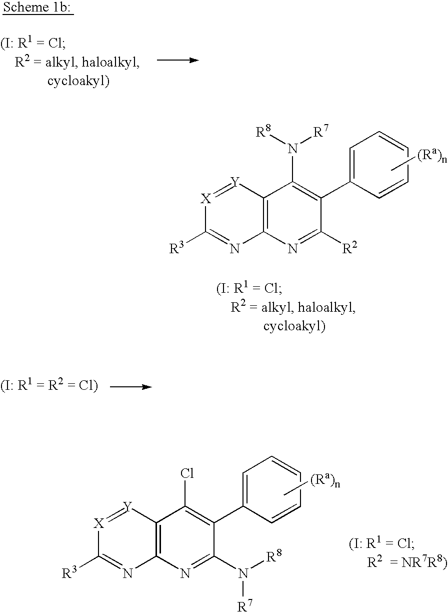

In scheme 1b, the variables n, R^{a}, R³, R⁷, R⁸, X and Y are as defined above.

The reaction of the monochloro compound I {R¹=Cl, R²=C₁-C₆-alkyl, C₁-C₆-haloalkyl or optionally substituted C₃-C₈-cycloalkyl} or the dichloro compounds I {R¹=R²=Cl} with an amine HNR⁷R⁸ is usually carried out at from 0 to 150° C., preferably at from 10 to 120° C., in an inert solvent, if appropriate in the presence of an auxiliary base. This method is known in principle, for example from II Farmaco, 57, 2002, 631, and can be applied in a manner analogous to the preparation of the compounds according to the invention.

Suitable solvents are protic solvents, such as alcohols, for example ethanol, and also aprotic solvents, for example aromatic hydrocarbons, halohydrocarbons and ethers, e.g. toluene, o-, m- and p-xylene, diethyl ether, diisopropyl ether, tert-butyl methyl ether, dioxane, tetrahydrofuran, dichloromethane, and also mixtures of the solvents mentioned above. Suitable auxiliary bases are, for example, those mentioned below: alkali metal carbonates and hydrogencarbonates, such as NaHCO₃ and Na₂CO₃, alkali metal hydrogenphosphates, such as Na₂HPO₄, alkali metal borates, such as Na₂B₄O₇, tertiary amines, such as triethylamine, ethyldiisopropylamine or diethylaniline, and pyridine compounds. A suitable auxiliary base is also an excess of the amine HNR⁷R⁸.

Usually, the components are employed in an approximately stoichiometric ratio. However, it can be advantageous to use an excess of amine HNR⁷R⁸. When using an excess of amine HNR⁷R⁸, the amine can simultaneously act as solvent. The amines HNR⁷R⁸ are commercially available or known from the literature, or they can be prepared by known methods.

Compounds of the formula I in which R² is O—C₁-C₄-alkylphenyl, where phenyl is optionally mono- or polysubstituted, can also be prepared from the corresponding dichloro compound I {R¹=Cl, R²=Cl} by reaction with an alcohol R⁶OH, as shown in scheme 1c. Such reactions are known in principle, for example from JACS, 69, 1947, 1204. The reaction is generally carried out in the presence of a base. Suitable bases are alkali metal hydrides, such as sodium hydride or potassium hydride, alkali metal or alkaline earth metal alkoxides, such as sodium t-butoxide or potassium tert-butoxide, or tertiary amines, such as triethylamine or pyridine. Alternatively, the alcohol R⁶OH can also initially be reacted with an alkali metal, preferably sodium, with formation of the corresponding alkoxide. The reaction can be carried out in excess alcohol or in an inert solvent, such as a carboxamide, for example N,N-dimethylformamide, N,N-dimethylacetamide or N-methylpyrrolidone. The reaction is usually carried out at from 0° C. to 150° C., preferably at from 10° C. to 100° C.

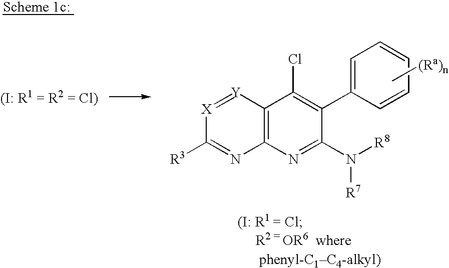

In scheme 1c, n, R^{a}, R³, X and Y are as defined above, and the phenyl radical in R⁶ may optionally be mono- or polysubstituted by alkyl, alkoxy or halogen.

Compounds of the formula I in which R¹ is NR⁷R⁸ and R² is halogen, in particular chlorine, can be obtained, for example, from the corresponding halogen compounds of the formula I {R¹=halogen, R²=OR⁶, where R⁶=phenyl-C₁-C₄-alkyl). The reaction sequence is shown in scheme 1d for the preparation of compounds I where R¹=NR⁷R⁸ and R²=Cl. In scheme 1d, n, R^{a}, R³, R⁷, R⁸, X and Y are as defined above, and the phenyl radical in R⁶ may optionally be mono- or polysubstituted by alkyl, alkoxy or halogen.

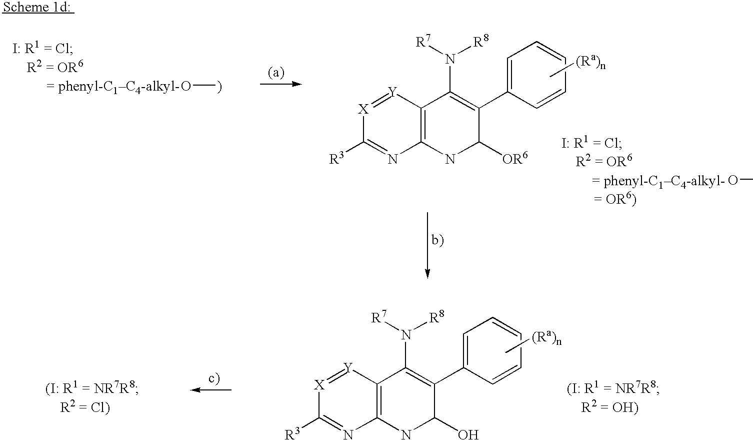

The reaction shown in step a) can be carried out in a known manner, for example analogously to the method shown in scheme 1b. In the resulting compound I {R¹=NR⁷R⁸ and R²=OR⁶, where R⁶=phenyl-C₁-C₄-alkyl}, the ether bond can be cleaved by catalytic hydrogenolysis, for example according to the method described in Org. Lett., 3, 2001, 4263. Suitable catalysts are, for example, noble metals or transition metals, such as palladium or platinum. In general, the catalyst is supported, for example on activated carbon. The hydrogenolysis is usually carried out in a solvent. Suitable solvents are, for example, alcohols, such as methanol, or cyclic ethers, such as tetrahydrofuran or dioxane. In general, the hydrogenolysis is carried out under atmospheric pressure. The hydrogenolysis is generally carried out at temperatures between room temperature and the boiling point of the solvent, preferably at temperatures between room temperature and 40° C.

The compounds of the formula I obtained in this manner where R¹=NR⁷R⁸ and R²=OH can then be converted into the corresponding compounds where R¹=NR⁷R⁸ and R²=halogen, preferably chlorine. Processes for converting alcohols into the corresponding halides are known from the prior art, for example from J. Chem. Soc. 1947, 899. Suitable halogenating agents are, for example, the halogenating agents mentioned above (see scheme 1a). The reaction can be carried out in excess halogenating agent, for example POCl₃, or in an inert solvent, such as acetonitrile or 1,2-dichloroethane.

The reaction is generally carried out between 10 and 180° C., preferably between room temperature and 130° C.

Compounds of the formula I in which R² is cyano can be obtained from the corresponding chloro compounds of the formula I {R¹=NR⁷R⁸, R²=Cl} by reaction with alkali metal, alkaline earth metal or metal cyanides, such as NaCN, KCN or Zn(CN)₂ [cf.: Heterocycles, Vol. 39, pp. 345-356 (1994); Collect. Czech. Chem. Commun. Vol. 60, pp. 1386-1389 (1995); Acta Chim. Scand., Vol. 50, pp. 58-63 (1996)].

The conversion of chloro compounds of the formula I {R¹=NR⁷R⁸, R²=Cl} into compounds of the formula I in which R² is C₁-C₆-alkyl, C₁-C₆-haloalkyl, C₂-C₆-alkenyl, C₂-C₆-alkynyl, optionally substituted C₃-C₈-cycloalkyl or optionally substituted C₅-C₈-cycloalkenyl can be carried out in a manner known per se by reacting organometallic compounds R^{2a}-Met in which R², is C₁-C₆-alkyl, C₁-C₆-haloalkyl, C₂-C₆-alkenyl, C₂-C₆-alkynyl, optionally substituted C₃-C₈-cycloalkyl or optionally substituted C₅-C₈-cycloalkenyl and Met is lithium, magnesium or zinc. The reaction is preferably carried out in the presence of catalytic or, in particular, at least equimolar amounts of transition metal salts and/or compounds, in particular in the presence of Cu salts, such as Cu(I) halides and especially Cu(I) iodide. In general, the reaction is carried out in an inert organic solvent, for example an ether, in particular tetrahydrofuran, an aliphatic or cycloaliphatic hydrocarbon, such as hexane, cyclohexane and the like, an aromatic hydrocarbon, such as toluene, or in a mixture of these solvents. The required temperatures are in the range from −100 to +100° C. and especially in the range from −80° C. to +40° C.

Analogously, it is possible to convert chloro compounds of the formula I {R¹=Cl, R²=NR⁷R⁸}, in which R¹ is C₁-C₆-alkyl, C₁-C₆-haloalkyl, C₂-C₆-alkenyl, C₂-C₆-alkynyl, C₃-C₈-cycloalkyl or C₅-C₈-cycloalkenyl.

Compounds of the formula I in which R¹ is C₁-C₆-alkyl, C₁-C₆-haloalkyl, C₂-C₆-alkenyl, C₂-C₆-alkynyl, C₃-C₈-cycloalkyl or C₅-C₈-cycloalkenyl can be prepared, for example, by reacting the chloro compound I {R¹=Cl and R²=alkyl, cycloalkyl) in the manner described above with organometallic compounds R^{1a}, where R^{1a} is C₁-C₆-alkyl, C₁-C₆-haloalkyl, C₂-C₆-alkenyl, C₂-C₆-alkynyl, optionally substituted C₃-C₈-cycloalkyl or optionally substituted C₅-C₈-cycloalkyl and Met is lithium, magnesium or zinc.

By appropriate modification of the synthesis shown in scheme 1b, it is possible to introduce, instead of the group NR⁷R⁸, a nitrile group, a group OR^{6′} {R^{6′}=alkyl} or a group S—R^{6″} {R^{6″}=H or alkyl} as substituent R¹ into compounds I where R¹=Cl and R²=alkyl, haloalkyl or optionally substituted alkyl, using the methods mentioned here.

Some of the hetarylamines of the formula II are commercially available or known from the literature, or they can be prepared analogously to processes known from the literature, for example J. Chem. Soc. 1937, 367; J. Chem. Soc. 1953, 331; Bioorg. Med. Chem. 9, (2001) 2061; JACS 67, 1945, 1711.

Substituted phenylacetic esters of the formula III are known from the literature or can be prepared analogously to known processes.

The ketones of the formula III used as starting materials are useful starting materials for preparing the compounds I according to the invention {R²=C₁-C₆-alkyl, C₁-C₆-haloalkyl, C₂-C₆-alkenyl, C₂-C₆-alkynyl, C₃-C₈-cycloalkyl which is optionally mono- or polysubstituted by alkyl and/or halogen, C₅-C₈-cycloalkenyl which is optionally mono- or polysubstituted by alkyl and/or halogen}.

The ketones of the formula III are novel if the radical 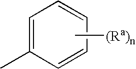
is a radical of the formula 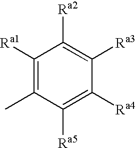
in which
R^{a1} is fluorine, chlorine, trifluoromethyl or methyl;
R^{a2} is hydrogen or fluorine;
R^{a3} is hydrogen, fluorine, chlorine, cyano, C₁-C₄-alkyl, especially methyl, C₁-C₄-alkoxy, especially methoxy, or C₁-C₄-alkoxycarbonyl, especially methoxycarbonyl;
R^{a4} is hydrogen, chlorine or fluorine;
R^{a5} is hydrogen, fluorine, chlorine or C₁-C₄-alkyl, especially methyl, or C₁-C₄-alkoxy, especially methoxy.

Accordingly, the present invention also provides ketones of the formula IIID 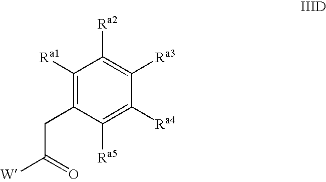
in which
R^{a1}, R^{a2}, R^{a3}, R^{a4} and R^{a5} are as defined above and
W′ is C₁-C₆-alkyl, C₁-C₆-haloalkyl, C₂-C₆-alkenyl, C₂-C₆-alkynyl, C₃-C₈-cycloalkyl which is optionally mono- or polysubstituted by alkyl and/or halogen, C₅-C₈-cycloalkenyl which is optionally mono- or polysubstituted by alkyl and/or halogen.

Preferably, W′ is C₁-C₆-alkyl, in particular methyl. Preferably, at least one of the radicals R^{a3} or R^{a5} is different from hydrogen. Preferably, at least one and particularly preferably both radicals R^{a2}, R^{a4} are hydrogen.

In a very particularly preferred embodiment of the present invention, (R^{a})ₙ is 2-CH₃-4-Cl, 2-F-4-CH₃, 2,6-di-F-4-CH₃, 2,6-di-F-4-CN, 2,6-di-F-4-COOCH₃, 2-CF₃-4-F, 2-CF₃-5-F, 2-CF₃-5-Cl, 2-F-6-CH₃, 2,6-di-F-4-OCH₃, 2-CH₃-4-F, 2-F-6-OCH₃, 2-F-4-Cl, 2-Cl-4-F, 2,5-di-F, 2,4,6-tri-F or 2,3,4-tri-F.

The ketones of the formula III, in particular the ketones of the formula IIID, can be prepared, for example, according to scheme 2 by heating a phenyl-β-keto ester of the formula IV in the presence of a weak aqueous acid, for example lithium chloride.

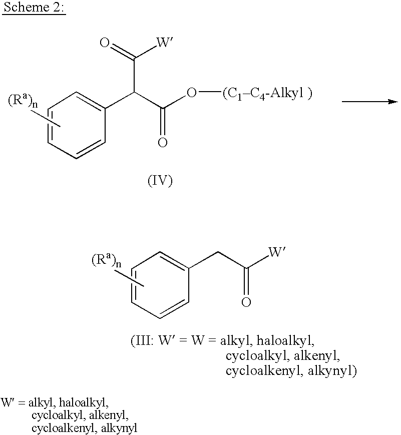

In scheme 2, R^{a} and n have the meanings mentioned above, in particular the meanings mentioned as being preferred, W′ is C₁-C₆-alkyl, C₁-C₆-haloalkyl, optionally substituted C₃-C₈-cycloalkyl, optionally substituted C₅-C₈-cycloalkenyl, C₂-C₆-alkenyl or C₂-C₆-alkynyl. In a preferred embodiment of the present invention, the compound IV is employed as its ethyl ester.

In general, the acid is employed in excess, based on the phenyl-β-keto ester IV. Usually, the phenyl-β-keto ester IV is heated in a solvent. Suitable solvents are dipolar aprotic solvents, such as dimethyl sulfoxide. The reaction temperature is usually in the range from room temperature to the boiling point of the solvent, preferably in the range from 60° C. to the boiling point of the solvent. The phenyl-β-keto esters IV are either known from the literature, for example from WO 99/41255, or they can be prepared analogously to processes known from the literature, for example analogously to Houben-Weyl, Volume VII/2a, p. 521.

The compounds I are suitable as fungicides. They are distinguished by an outstanding effectiveness against a broad spectrum of phytopathogenic fungi, especially from the class of the Ascomycetes, Deuteromycetes, Oomycetes and Basidiomycetes. Some are systemically effective and can be used in crop protection as foliar and soil fungicides.

They are particularly important in the control of a multitude of fungi on various cultivated plants, such as wheat, rye, barley, oats, rice, corn, grass, bananas, cotton, soybean, coffee, sugar cane, vines, fruits and ornamental plants, and vegetables, such as cucumbers, beans, tomatoes, potatoes and cucurbits, and on the seeds of these plants.

They are especially suitable for controlling the following plant diseases:

Alternaria species on fruit and vegetables,

Bipolaris and Drechslera species on cereals, rice and lawns,
Blumeria graminis (powdery mildew) on cereals,
Botrytis cinerea (gray mold) on strawberries, vegetables, ornamental plants and grapevines,
Erysiphe cichoracearum and Sphaerotheca fuliginea on cucurbits,
Fusarium and Verticillium species on various plants,
Mycosphaerella species on cereals, bananas and peanuts,
Phytophthora infestans on potatoes and tomatoes,
Plasmopara viticola on grapevines,
Podosphaera leucotricha on apples,
Pseudocercosporella herpotrichoides on wheat and barley,
Pseudoperonospora species on hops and cucumbers,
Puccinia species on cereals,
Pyricularia oryzae on rice,
Rhizoctonia species on cotton, rice and lawns,
Rhynchosporium secalis (leaf blotch) on cereals,
Septoria tritici and Stagonospora nodorum on wheat,
Uncinula necator on grapevines,
Ustilago species on cereals and sugar cane, and
Venturia species (scab) on apples and pears.

The compounds I are also suitable for controlling harmful fungi, such as Paecilomyces variotii, in the protection of materials (e.g. wood, paper, paint dispersions, fibers or fabrics) and in the protection of stored products.

The compounds I are employed by treating the fungi or the plants, seeds, materials or soil to be protected from fungal attack with a fungicidally effective amount of the active compounds. The application can be carried out both before and after the infection of the materials, plants or seeds by the fungi.

The fungicidal compositions generally comprise between 0.1 and 95%, preferably between 0.5 and 90%, by weight of active compound.

When employed in crop protection, the amounts applied are, depending on the kind of effect desired, between 0.01 and 2.0 kg of active compound per ha.

In seed treatment, amounts of active compound of 0.001 to 1 g, preferably 0.01 to 0.05 g, per kilogram of seed are generally required.

When used in the protection of materials or stored products, the amount of active compound applied depends on the kind of application area and on the desired effect. Amounts customarily applied in the protection of materials are, for example, 0.001 g to 2 kg, preferably 0.005 g to 1 kg, of active compound per cubic meter of treated material.

The compounds I can be converted into the customary formulations, for example solutions, emulsions, suspensions, dusts, powders, pastes and granules. The application form depends on the particular purpose; in each case, it should ensure a fine and uniform distribution of the compound according to the invention.

The formulations are prepared in a known way, e.g. by extending the active compound with solvents and/or carriers, if desired using emulsifiers and dispersants, it being possible, when water is the diluent, also to use other organic solvents as auxiliary solvents. Suitable auxiliaries for this purpose are essentially: solvents, such as aromatics (e.g. xylene), chlorinated aromatics (e.g. chlorobenzenes), paraffins (e.g. petroleum fractions), alcohols (e.g. methanol, butanol), ketones (e.g. cyclohexanone), amines (e.g. ethanolamine, dimethylformamide) and water; carriers, such as ground natural minerals (e.g. kaolins, clays, talc, chalk) and ground synthetic minerals (e.g. highly dispersed silica, silicates); emulsifiers, such as nonionic and anionic emulsifiers (e.g. polyoxyethylene fatty alcohol ethers, alkylsulfonates and arylsulfonates) and dispersants, such as lignosulfite waste liquors and methylcellulose.

Suitable surfactants are alkali metal, alkaline earth metal and ammonium salts of lignosulfonic acid, naphthalenesulfonic acid, phenolsulfonic acid and dibutylnaphthalenesulfonic acid, alkylarylsulfonates, alkyl sulfates, alkylsulfonates, fatty alcohol sulfates and fatty acids, and alkali metal and alkaline earth metal salts thereof, salts of sulfated fatty alcohol glycol ethers, condensation products of sulfonated naphthalene and naphthalene derivatives with formaldehyde, condensation products of naphthalene or of naphthalenesulfonic acid with phenol and formaldehyde, polyoxyethylene octylphenol ethers, ethoxylated isooctylphenol, octylphenol and nonylphenol, alkylphenol polyglycol ethers, tributylphenyl polyglycol ethers, alkylaryl polyether alcohols, isotridecyl alcohol, fatty alcohol ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Mineral oil fractions having medium to high boiling points, such as kerosene or diesel fuel, furthermore coal tar oils, and oils of vegetable or animal origin, aliphatic, cyclic and aromatic hydrocarbons, e.g. benzene, toluene, xylene, paraffin, tetrahydronaphthalene, alkylated naphthalenes or derivatives thereof, methanol, ethanol, propanol, butanol, chloroform, carbon tetrachloride, cyclohexanol, cyclohexanone, chlorobenzene or isophorone, or highly polar solvents, e.g. dimethylformamide, dimethyl sulfoxide, N-methylpyrrolidone or water, are suitable for the preparation of directly sprayable solutions, emulsions, pastes or oil dispersions.

Powders, preparations for broadcasting and dusts can be prepared by mixing or grinding the active substances together with a solid carrier.

Granules, e.g. coated granules, impregnated granules and homogeneous granules, can be prepared by binding the active compounds to solid carriers. Solid carriers are, e.g., mineral earths, such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, e.g., ammonium sulfate, ammonium phosphate, ammonium nitrate or ureas, and plant products, such as cereal meal, tree bark meal, wood meal and nutshell meal, cellulose powders and other solid carriers.

The formulations generally comprise between 0.01 and 95% by weight, preferably between 0.1 and 90% by weight, of the active compound. The active compounds are employed therein in a purity of 90% to 100%, preferably 95% to 100% (according to the NMR spectrum).

Examples for formulations are:
I. 5 parts by weight of a compound according to the invention are intimately mixed with 95 parts by weight of finely divided kaolin. In this way, a dust comprising 5% by weight of the active compound is obtained.
II. 30 parts by weight of a compound according to the invention are intimately mixed with a mixture of 92 parts by weight of pulverulent silica gel and 8 parts by weight of liquid paraffin, which had been sprayed onto the surface of this silica gel. In this way, an active compound preparation with good adhesive properties (active compound content 23% by weight) is obtained.
III. 10 parts by weight of a compound according to the invention are dissolved in a mixture consisting of 90 parts by weight of xylene, 6 parts by weight of the addition product of 8 to 10 mol of ethylene oxide with 1 mol of the N-monoethanolamide of oleic acid, 2 parts by weight of the calcium salt of dodecylbenzenesulfonic acid and 2 parts by weight of the addition product of 40 mol of ethylene oxide with 1 mol of castor oil (active compound content 9% by weight).
IV. 20 parts by weight of a compound according to the invention are dissolved in a mixture consisting of 60 parts by weight of cyclohexanone, 30 parts by weight of isobutanol, 5 parts by weight of the addition product of 7 mol of ethylene oxide with 1 mol of isooctylphenol and 5 parts by weight of the addition product of 40 mol of ethylene oxide with 1 mol of castor oil (active compound content 16% by weight).
V. 80 parts by weight of a compound according to the invention are intimately mixed with 3 parts by weight of the sodium salt of diisobutylnaphthalene-α-sulfonic acid, 10 parts by weight of the sodium salt of a lignosulfonic acid from a sulfite waste liquor and 7 parts by weight of pulverulent silica gel and are ground in a hammer mill (active compound content 80% by weight).
VI. 90 parts by weight of a compound according to the invention are mixed with 10 parts by weight of N-methyl-α-pyrrolidone and a solution is obtained which is suitable for use in the form of very small drops (active compound content 90% by weight).
VII. 20 parts by weight of a compound according to the invention are dissolved in a mixture consisting of 40 parts by weight of cyclohexanone, 30 parts by weight of isobutanol, 20 parts by weight of the addition product of 7 mol of ethylene oxide with 1 mol of isooctylphenol and 10 parts by weight of the addition product of 40 mol of ethylene oxide with 1 mol of castor oil. By pouring the solution into 100 000 parts by weight of water and finely dispersing it therein, an aqueous dispersion is obtained comprising 0.02% by weight of the active compound.
VIII. 20 parts by weight of a compound according to the invention are intimately mixed with 3 parts by weight of the sodium salt of diisobutylnaphthalene-α-sulfonic acid, 17 parts by weight of the sodium salt of a lignosulfonic acid from a sulfite waste liquor and 60 parts by weight of pulverulent silica gel and are ground in a hammer mill. A spray emulsion comprising 0.1% by weight of the active compound is obtained by fine dispersion of the mixture in 20 000 parts by weight of water.

The active compounds can be used as such, in the form of their formulations or of the application forms prepared therefrom, e.g. in the form of directly sprayable solutions, powders, suspensions or dispersions, emulsions, oil dispersions, pastes, dusts, preparations for broadcasting or granules, by spraying, atomizing, dusting, broadcasting or watering. The application forms depend entirely on the intended uses; they should always ensure the finest possible dispersion of the active compounds according to the invention.

Aqueous use forms can be prepared from emulsifiable concentrates, pastes or wettable powders (spray powders, oil dispersions) by addition of water. To prepare emulsions, pastes or oil dispersions, the substances can be homogenized in water, as such or dissolved in an oil or solvent, by means of wetting agents, tackifiers, dispersants or emulsifiers. However, concentrates comprising active substance, wetting agent, tackifier, dispersant or emulsifier and possibly solvent or oil can also be prepared and are suitable for dilution with water.

The concentrations of active compound in the ready-for-use preparations can be varied within relatively wide ranges. In general, they are between 0.0001 and 10%, preferably between 0.01 and 1%.

The active compounds can also be used with good success in the ultra low volume (ULV) process, it being possible to apply formulations with more than 95% by weight of active compound or even the active compound without additives.

Oils of various types, herbicides, fungicides, other pesticides and bactericides can be added to the active compounds, if appropriate even not until immediately before use (tank mix). These agents can be added to the preparations according to the invention in a weight ratio of 1:10 to 10:1.

The preparations according to the invention can, in the application form as fungicides, also be present together with other active compounds, e.g. with herbicides, insecticides, growth regulators, fungicides or also with fertilizers. On mixing the compounds I or the preparations comprising them in the application form as fungicides with other fungicides, in many cases an expansion of the fungicidal spectrum of activity is obtained.

The following list of fungicides, with which the compounds according to the invention can be used in conjunction, is intended to illustrate the possible combinations but not to limit them:
acylalanines, such as benalaxyl, metalaxyl, ofurace or oxadixyl,
amine derivatives, such as aldimorph, dodine, dodemorph, fenpropimorph, fenpropidin, guazatine, iminoctadine, spiroxamine or tridemorph,
anilinopyrimidines, such as pyrimethanil, mepanipyrim or cyprodinil,
antibiotics, such as cycloheximide, griseofulvin, kasugamycin, natamycin, polyoxin or streptomycin,
azoles, such as bitertanol, bromoconazole, cyproconazole, difenoconazole, dinitroconazole, epoxiconazole, fenbuconazole, fluquinconazole, flusilazole, hexaconazole, imazalil, metconazole, myclobutanil, penconazole, propiconazole, prochloraz, prothioconazole, tebuconazole, triadimefon, triadimenol, triflumizole or triticonazole,
dicarboximides, such as iprodione, myclozolin, procymidone or vinclozolin,
dithiocarbamates, such as ferbam, nabam, maneb, mancozeb, metam, metiram, propineb, polycarbamate, thiram, ziram or zineb,
heterocyclic compounds, such as anilazine, benomyl, boscalid, carbendazim, carboxin, oxycarboxin, cyazofamid, dazomet, dithianon, famoxadone, fenamidone, fenarimol, fuberidazole, flutolanil, furametpyr, isoprothiolane, mepronil, nuarimol, probenazole, proquinazid, pyrifenox, pyroquilon, quinoxyfen, silthiofam, thiabendazole, thifluzamide, thiophanate-methyl, tiadinil, tricyclazole or triforine,
copper fungicides, such as Bordeaux mixture, copper acetate, copper oxychloride or basic copper sulfate,
nitrophenyl derivatives, such as binapacryl, dinocap, dinobuton or nitrophthal-isopropyl,
phenylpyrroles, such as fenpiclonil or fludioxonil,
sulfur,
other fungicides, such as acibenzolar-S-methyl, benthiavalicarb, carpropamid, chlorothalonil, cyflufenamid, cymoxanil, dazomet, diclomezine, diclocymet, diethofencarb, edifenphos, ethaboxam, fenhexamid, fentin acetate, fenoxanil, ferimzone, fluazinam, fosetyl, fosetyl-aluminum, iprovalicarb, hexachlorobenzene, metrafenone, pencycuron, propamocarb, phthalide, tolclofos-methyl, quintozene or zoxamide,
strobilurins, such as azoxystrobin, dimoxystrobin, fluoxastrobin, kresoxim-methyl, metominostrobin, orysastrobin, picoxystrobin, pyraclostrobin or trifloxystrobin,
sulfenic acid derivatives, such as captafol, captan, dichlofluanid, folpet or tolylfluanid,
cinnamides and analogous compounds, such as dimethomorph, flumetover or flumorph.

### SYNTHESIS EXAMPLES

The procedures described in the following synthesis examples were used to prepare further compounds I by appropriate modification of the starting compounds. The compounds thus obtained are listed in the following table, together with physical data.

### Precursor Example 1

### 1-(2,4,6-Trifluorophenyl)propan-2-one

A little at a time, 42.4 g (0.103 mol) of lithium chloride were added to 15 g (0.052 mol) of ethyl 3-oxo-2-(2,4,6-trifluorophenyl)butanoate in 100 ml of dimethyl sulfoxide. 18 g (0.052 mol) of water were then added dropwise, and the resulting reaction mixture was stirred at 110° C. for 6.5 hours. The reaction mixture was cooled, 50 ml of water were added and the aqueous reaction mixture was extracted repeatedly with cyclohexane. The combined organic phases were dried, the drying agent was then filtered off and the filtrate was concentrated. The residue obtained was chromatographed on silica gel (cyclohexane/ethyl acetate 99:1), which gave, after concentration, 4.5 g (46%) of the title compound.

In an analogous manner, it is possible to obtain the compounds III listed in Table 1 below:

TABLE 1

| | |
|---|---|
| | (III) |
| 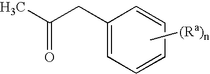 | |

| Precursor example | (R^{a})ₙ |
|---|---|
| Precursor example 2 | 2-CH₃-4-Cl |
| Precursor example 3 | 2-F-4-CH₃ |
| Precursor example 4 | 2,6-di-F-4-CH₃ |
| Precursor example 5 | 2,6-di-F-4-CN |
| Precursor example 6 | 2,6-di-4-COOCH₃ |
| Precursor example 7 | 2-CF₃-4-F |
| Precursor example 8 | 2-CF₃-5-F |
| Precursor example 9 | 2-CF₃-5-Cl |
| Precursor example 10 | 2-F-6-CH₃ |
| Precursor example 11 | 2,6-di-F-4-OCH₃ |
| Precursor example 12 | 2-CH₃-4-F |
| Precursor example 13 | 2-F-6-OCH₃ |
| Precursor example 14 | 2-F-4-Cl |
| Precursor example 15 | 2-Cl-4-F |
| Precursor example 16 | 2,5-di-F |
| Precursor example 17 | 2,3,4-tri-F |

### Example 1

### 6-(2,4,6-Trifluorophenyl)pyrido[2,3-d]pyrimidine-5,7-diol

At room temperature, 2.44 g (0.036 mol) of sodium ethoxide were added to 6.7 g (0.033 mol) of ethyl 2,4,6-trifluorophenylacetate, and the mixture was stirred for about 5 minutes. 3 g (0.018 mol) of ethyl 4-aminopyrimidine-5-carboxylate were then added, and the resulting suspension was heated to 130° C. To improve the stirrability of the suspension, a further 8 ml of ethyl 2,4,6-trifluorophenylacetate were added. The suspension was heated for 6 hours, and the ethanol formed was distilled off. The reaction mixture was then allowed to cool and poured into water.

The aqueous reaction mixture was extracted with ethyl acetate. The organic layer was dried, the drying agent was filtered off and the filtrate was evaporated to dryness, which resulted in the recovery of 8.6 g of ethyl 2,4,6-trifluorophenylacetate. Using acetic acid, the aqueous phase was adjusted to a pH of 5.5, which resulted in the precipitation of a solid. The precipitated solid was filtered off and dried, which gave 1.6 g (30%) of the title compound.

### Example 2

### 5,7-Dichloro-6-(2,4,6-trifluorophenyl)pyrido[2,3-d]pyrimidine

With stirring, 7.74 g (0.037 mol) of phosphorus pentachloride were added to 4.2 g (0.014 mol) of 6-(2,4,6-trifluorophenyl)pyrido[2,3-d]pyrimidine-5,7-diol from Example 1 in 40 ml of phosphorus oxychloride, and the mixture was heated with stirring at 130° C. for 8 hours. After cooling, the reaction mixture was concentrated and the residue was taken up in dichloromethane. 150 ml of water were then added cautiously, and the aqueous reaction mixture was made alkaline using sodium carbonate solution. The organic phase was separated off, the aqueous phase was extracted twice with dichloromethane and the combined organic phases were dried. Removal of the drying agent by filtration and concentration of the organic phase gave 4.4 g (95%) of the title compound.

### Example 3

### 7-Benzyloxy-5-chloro-6-(2,4,6-trifluorophenyl)pyrido[2,3-d]pyrimidine

With cooling, 10 ml of benzyl alcohol were cautiously added dropwise to 0.29 g (0.0073 mol) of 60% pure sodium hydride in white mineral oil, and the mixture was stirred at room temperature for 30 minutes. 2.4 g (0.0073 mol) of 5,7-dichloro-6-(2,4,6-trifluorophenyl)pyrido[2,3-d]pyrimidine from Example 2 were then cautiously added dropwise, and the mixture was allowed to stand at room temperature for about 100 hours. The reaction mixture was poured into water and extracted three times with ethyl acetate, and the combined organic phases were dried. Removal of the drying agent by filtration and concentration of the organic phase gave 3 g (100%) of the title compound, which was slightly contaminated.

### Example 4

### 5-Chloro-7-(4-methylpiperidinyl)-6-(2,4,6-trifluorophenyl)pyrido[2,3-d]pyrimidine

0.1 g (0.3 mmol) of 5,7-dichloro-6-(2,4,6-trifluorophenyl)pyrido[2,3-d]pyrimidine from Example 2 was dissolved in 1 ml of dichloromethane and 0.04 ml of triethylamine. 0.03 g (3 mmol) of 4-methylpiperidine was then added, and the mixture was stirred at room temperature for 12 hours. The reaction mixture was taken up in a little water and dichloromethane, and the organic phase was washed with dilute aqueous hydrochloric acid. After drying of the organic phase, the drying agent was filtered off and the filtrate was evaporated to dryness, which gave 0.1 g (85%) of the title compound.

### Example 5

### 2,7-Dimethyl-3-(2,4,6-trifluorophenyl)-[1,8]naphthyridin-4-ol

At 120° C., 1.3 g (0.0072 mol) of ethyl 2-amino-6-methylnicotinate and 1.48 g (0.0079 mol) of 1-(2,4,6-trifluorophenyl)propan-2-one from precursor example 1 were, a little at a time and alternately, added to 5 g of polyphosphoric acid, and, after the addition had ended, the mixture was heated at 150° C. for another 5 hours. The reaction mixture was allowed to cool, then about 60 ml of ice-water were added and the mixture was adjusted to pH 7 using 4 N sodium hydroxide solution, which resulted in the formation of a precipitate. The precipitate was filtered off and dried, which gave 0.56 g of the title compound.

### Example 6

### 4-Chloro-2,7-dimethyl-3-(2,4,6-trifluorophenyl)-[1,8]naphthyridine

0.147 g (0.7 mmol) of phosphorus pentachloride was added to 0.165 g (0.54 mmol) of 2,7-dimethyl-3-(2,4,6-trifluorophenyl)-[1,8]naphthyridin-4-ol from Example 5 in 1.26 ml of phosphorus oxychloride. The resulting reaction mixture was stirred at 110° C. for 3 hours and then allowed to cool, ice-water was added and the mixture was made alkaline using aqueous sodium carbonate solution. The aqueous reaction mixture was extracted four times with dichloromethane, the combined organic phases were dried, the drying agent was filtered off and the filtrate was concentrated, which gave 0.18 g of the title compound.

### Example 7

### 7-Benzyloxy-5-(4-methylpiperidinyl)-6-(2,4,6-trifluorophenyl)pyrido[2,3-d]-pyrimidine and 5-chloro-6-(2,4,6-trifluorophenyl)pyrido[2,3-d]pyrimidin-7-ol

The reaction mixture of 0.12 g (0.3 mmol) of 7-benzyloxy-5-chloro-6-(2,4,6-trifluorophenyl)pyrido[2,3-d]pyrimidine from Example 3, 1.5 g of 4-methylpiperidine and 0.05 g of triethylamine was stirred at 80° C. for 3 hours. The reaction mixture was taken up in water, and the aqueous mixture was washed three times with dichloromethane. The combined organic phases were dried, then the drying agent was filtered off and the filtrate was concentrated. The residue was chromatographed on silica gel using cyclohexane:ethyl acetate (95:5), which gave 0.06 g of a mixture of the title compound and 5-chloro-6-(2,4,6-trifluorophenyl)pyrido[2,3-d]pyrimidin-7-ol.

### Example 8

### 5-(4-Methylpiperidin-1-yl)-6-(2,4,6-trifluorophenyl)pyrido[2,3-d]pyrimidin-7-ol

0.6 g (0.0013 mol) of 7-benzyloxy-5-(4-methylpiperidin-1-yl)-6-(2,4,6-trifluorophenyl)pyrido[2,3-d]pyrimidine (80% pure) from Example 7 in 20 ml of methanol was initially charged in a laboratory hydrogenation apparatus with aerator-stirrer (2000 min⁻¹). 0.03 g of Pd/C (10%) was then added, and the mixture was hydrogenated until the maximum amount of hydrogen had been taken up. At a temperature of 30° C., the mixture was hydrogenated for about 90 min. The mixture was then filtered off with suction through kieselguhr, and the residue obtained was concentrated under reduced pressure. Purification by column chromatography gave 0.25 g (52%) of the title compound.

### Example 9

### 7-Chloro-5-(4-methylpiperidin-1-yl)-6-(2,4,6-trifluorophenyl)pyrido[2,3-d]pyrimidine

0.09 g (0.43 mmol) of phosphorus pentachloride was added to 0.1 g (2.67 mmol) of 5-(4-methylpiperidin-1-yl)-6-(2,4,6-trifluorophenyl)pyrido[2,3-d]pyrimidin-7-ol in 1.5 ml of phosphorus oxychloride, and the mixture was stirred at 120° C. for 5 hours. The reaction mixture was then poured into water and made alkaline using aqueous sodium carbonate solution, and the aqueous mixture was extracted with ethyl acetate. The organic phase was separated off and dried, the drying agent was filtered off and the filtrate was concentrated. The residue obtained was chromatographed on silica gel (cyclohexane/ethyl acetate), which gave, after evaporation to dryness, 0.05 g (48%) of the title compound.

The compounds of the formulae I.a, I.b and I.c listed in Tables 2 to 9 below were obtained in an analogous manner.

TABLE 2

| | |
|---|---|
| | (I.a) |
| 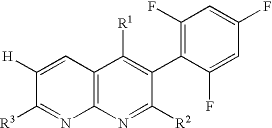 | |

| | | | | Melting point | |
|---|---|---|---|---|---|
| Example | R³ | R¹ | R² | [° C.] | MS [M⁺] |
| 5 | CH₃ | OH | CH₃ | 315 | |
| 6 | CH₃ | Cl | CH₃ | | 323 |
| 10 | CH₃ | OH | OH | 287 | |
| | | | | (decomposition) | |
| 11 | CH₃ | Cl | Cl | 208 | |
| 12 | H | OH | OH | 345 | |
| 13 | H | Cl | Cl | 115 | |
| 14 | CH₃ | Cl | C₆H₅CH₂O | | |
| 15 | H | OH | CH₃ | 313 | |
| 16 | H | Cl | C₆H₅CH₂O | 100 | |
| 17 | CHCl₂ | Cl | Cl | 141 | |
| 18 | CCl₃ | Cl | Cl | 81 | |
| 19 | H | Cl | CHCl₂ | | |

TABLE 3

| | |
|---|---|
| | (I.a) |
| 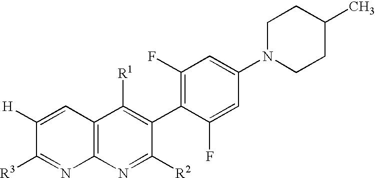 | |

| Example | R³ | R¹ | R² | Melting point [° C.] | MS [M⁺] |
|---|---|---|---|---|---|
| 20 | CH₃ | Cl | CH₃ | | |

TABLE 4

| | |
|---|---|
| | (I.b) |
| 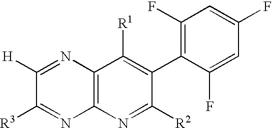 | |

| Example | R³ | R¹ | R² | Melting point [° C.] | MS [M⁺] |
|---|---|---|---|---|---|
| 21 | H | OH | OH | 253 | |
| 22 | H | Cl | OH | >330 | |
| 23 | H | Cl | Cl | | |

TABLE 5

| | |
|---|---|
| | (I.c) |
| 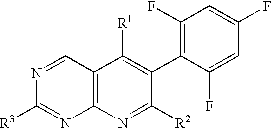 | |

| | | | | Melting point | |
|---|---|---|---|---|---|
| Example | R³ | R¹ | R² | [° C.] | MS [M⁺] |
| 1 | H | OH | OH | 348 | |
| | | | | (decomposition) | |
| 2 | H | Cl | Cl | 169 | |
| 3 | H | Cl | C₆H₅CH₂O | 146 | |
| 4 | H | Cl | 4-CH₃- | 125 | |
| | | | piperidinyl | | |
| 7 | H | 4-CH₃- | C₆H₅CH₂O | | 464 |
| | | piperidinyl | | | |
| 7 | H | Cl | OH | | 111 |
| 8 | H | 4-CH₃- | OH | 268 | |
| | | piperidinyl | | | |
| 9 | H | 4-CH₃- | Cl | | |
| | | piperidinyl | | | |
| 24 | H | Cl | S-CH₃ | 164 | |
| 25 | H | S-CH₃ | S-CH₃ | 104 | |

TABLE 6

| | |
|---|---|
| | (I.c) |
| 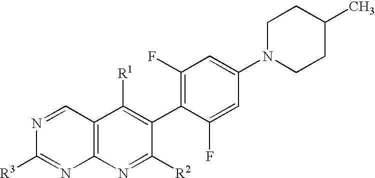 | |

| | | | | Melting point | |
|---|---|---|---|---|---|
| Example | R³ | R¹ | R² | [° C.] | MS [M⁺] |
| 26 | H | 4-CH₃-piperidinyl | OH | 251 | |

TABLE 7

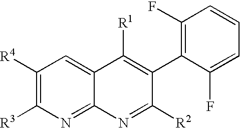

| No. | R³ | R¹ | R² | R⁴ | Physical data |
|---|---|---|---|---|---|
| 27 | H | OH | OH | H | 327° C. decomp. |
| 28 | H | Cl | Cl | H | 200° C. |
| 29 | H | Cl | Cl | Cl | 153° C. |
| 30 | H | Cl | OCH₂C₆H₅ | H | 108° C. |
| 31 | H | 4-CH₃-piperidine | OH | H | 296° C. |
| 32 | H | 4-CH₃-piperidine | Cl | H | 175° C. |
| 33 | H | Cl | OH | H | 270° C. |
| 34 | H | Cl | CH₃ | H | M⁺ 291 |
| 35 | H | OH | CH₃ | H | 260° C. decomp. |
| 36 | H | 4-CH₃-piperidine | CH₃ | H | M⁺ 353 |

TABLE 8

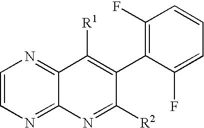

| No. | R¹ | R² | Physical data |
|---|---|---|---|
| 37 | Cl | 4-CH₃-piperidine | M⁺ 374.8 |

TABLE 9

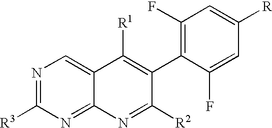

| No. | R³ | R¹ | R² | R | Physical data |
|---|---|---|---|---|---|
| 38 | H | 4-CH₃-piperidine | SCH₃ | F | 155° C. |
| 39 | H | OCH₃ | OCH₃ | F | 126° C. |
| 40 | H | Cl | OCH₃ | F | 125° C. |
| 41 | H | Cl | Cl | H | 1H NMR (CDCl₃: 9.87, |
| | | | | | 9.65 2H, pyrimidine-H |
| 42 | H | N(CH₃)₂ | Cl | F | M⁺ 338.8 |

### USE EXAMPLES

The active compounds were prepared as a stock solution with 0.25% by weight of active compound in acetone or DMSO (dimethyl sulfoxide). 1% by weight of the emulsifier Uniperol® EL (wetting agent having emulsifying and dispersing action based on ethoxylated alkylphenols) was added to this solution, and the solution was diluted with water to the desired concentration.

Activity against leaf blotch on wheat caused by Leptosphaeria nodorum, protective application.

Pots with wheat plants of the cultivar “Kanzler” were sprayed to runoff point with an aqueous suspension having the concentration of active compound stated below. The next day, the pots were inoculated with an aqueous spore suspension of Leptosphaeria nodorum. The plants were then placed in a chamber at 20° C. and maximum atmospheric humidity. After 8 days, the leaf blotch on the untreated but infected control plants had developed to such an extent that the degree of infection could be determined visually in %.

| Compound | Leaf infection [%] at 250 ppm |
|---|---|
| 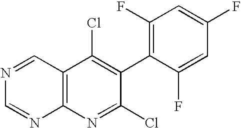 | 3 |
| 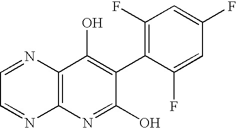 | 10 |
| 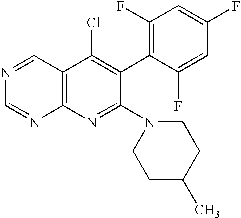 | 3 |
| untreated | 80 |

1. A bicyclic compound of the formula I 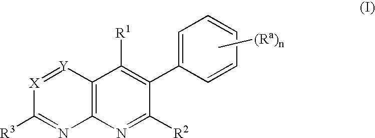
in which
X, Y independently of one another are N or C—R⁴;
n is 1, 2, 3, 4 or 5;
R^{a} is halogen, cyano, C₁-C₆-alkyl, C₁-C₆-alkoxy, C₁-C₆-haloalkyl, C₁-C₆-haloalkoxy, C₂-C₆-alkenyl, C₂-C₆-alkenyloxy or C(O)R⁵;
R¹ is halogen, cyano, C₁-C₆-alkyl, C₁-C₆-haloalkyl, C₂-C₆-alkenyl, C₂-C₆-alkynyl, C₃-C₈-cycloalkyl which is optionally mono- or polysubstituted by alkyl and/or halogen, C₅-C₈-cycloalkenyl which is optionally mono- or polysubstituted by alkyl and/or halogen, OR⁶, SR⁶ or NR⁷R⁸;
R² is halogen, cyano, C₁-C₆-alkyl, C₁-C₆-haloalkyl, C₂-C₆-alkenyl, C₂-C₆-alkynyl, C₃-C₈-cycloalkyl which is optionally mono- or polysubstituted by alkyl and/or halogen, C₅-C₈-cycloalkenyl which is optionally mono- or polysubstituted by alkyl and/or halogen, OR⁶, SR⁶ or NR⁷R⁸;
R³ is hydrogen, C₁-C₆-alkyl, C₁-C₆-haloalkyl or C₃-C₆-cycloalkyl which is optionally mono- or polysubstituted by alkyl and/or halogen;
R⁴ is hydrogen, halogen, C₁-C₆-alkyl, C₁-C₆-haloalkyl or C₃-C₆-cycloalkyl which is optionally mono- or polysubstituted by alkyl and/or halogen;
R⁵ is hydrogen, OH, C₁-C₆-alkyl, C₁-C₆-alkoxy, C₁-C₆-haloalkyl, C₁-C₆-haloalkoxy, C₂-C₆-alkenyl, C₁-C₆-alkylamino or di-C₁-C₆-alkylamino, piperidin-1-yl, pyrrolidin-1-yl or morpholin-4-yl;
R⁶ is hydrogen, C₁-C₆-alkyl, C₁-C₆-haloalkyl, phenyl-C₁-C₄-alkyl where phenyl may be mono- or polysubstituted by halogen, alkyl or alkoxy, C₂-C₆-alkenyl or COR⁹;
R⁷, R⁸ independently of one another are hydrogen, C₁-C₁₀-alkyl, C₂-C₁₀-alkenyl, C₄-C₁₀-alkadienyl, C₂-C₁₀-alkynyl, C₃-C₈-cycloalkyl, C₅-C₈-cycloalkenyl, C₅-C₁₀-bicycloalkyl, phenyl, phenyl-C₁-C₄-alkyl, naphthyl,
a 5- or 6-membered saturated or partially unsaturated heterocycle which may have 1, 2 or 3 heteroatoms selected from the group consisting of N, O and S as ring members, or
a 5- or 6-membered aromatic heterocycle which may have 1, 2 or 3 heteroatoms selected from the group consisting of N, O and S as ring members,
where the radicals mentioned as R⁷, R⁸ may be partially or fully halogenated and/or may have 1, 2 or 3 radicals R^{b}, where
R^{b} is selected from the group consisting of cyano, nitro, OH, C₁-C₆-alkyl, C₁-C₆-alkoxy, C₁-C₆-haloalkyl, C₁-C₆-haloalkoxy, C₁-C₆-alkylthio, C₂-C₆-alkenyl, C₂-C₆-alkenyloxy, C₂-C₆-alkynyl, C₂-C₆-alkynyloxy, C₁-C₆-alkylamino, di-C₁-C₆-alkylamino, piperidin-1-yl, pyrrolidin-1-yl or morpholin-4-yl;
R⁷ and R⁸ together with the nitrogen atom to which they are attached may also form a 5-, 6- or 7-membered saturated or unsaturated heterocycle which may have 1, 2, 3 or 4 further heteroatoms selected from the group consisting of O, S, N and NR¹⁰ as ring members and may be partially or fully halogenated and which may have 1, 2 or 3 radicals R^{b}; and
R⁹, R¹⁰ independently of one another are hydrogen or C₁-C₆-alkyl;
or an agriculturally acceptable salt of a compound I,
except for the compounds of the formula I in which R¹ is OH, if Y and X are simultaneously each C—R⁴;
and also except for 2,4-dichloro-3-(o-methoxyphenyl)-1,8-naphthyridine.

2. The compound according to claim 1 of the formula I in which Y and X are each C—R⁴.

3. The compound according to claim 1 of the formula I in which Y is N and X is C—R⁴.

4. The compound according to claim 1 of the formula I in which Y is C—R⁴ and X is N.

5. The compound according to any of the preceding claims of the formula I in which R⁴ is hydrogen, C₁-C₆-alkyl or C₁-C₆-haloalkyl.

6. The compound according to claim 1 of the formula I in which n is 2, 3, 4 or 5.

7. The compound according to claim 1 of the formula I in which the group 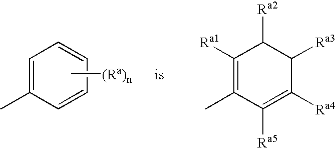
where
R^{a1} is fluorine, chlorine, trifluoromethyl or methyl;
R^{a2} is hydrogen or fluorine;
R^{a3} is hydrogen, fluorine, chlorine, cyano, C₁-C₄-alkyl, C₁-C₄-alkoxy or C₁-C₄-alkoxycarbonyl;
R^{a4} is hydrogen, chlorine or fluorine;
R^{a5} is hydrogen, fluorine, chlorine, C₁-C₄-alkyl or C₁-C₄-alkoxy.

8. The compound according to claim 1 of the formula I in which R¹ is a group NR⁷R⁸ in which at least one of the radicals R⁷, R⁸ is different from hydrogen.

9. The compound according to claim 8 of the formula I in which
R⁷ is C₁-C₆-alkyl, C₃-C₈-cycloalkyl which is optionally mono- or polysubstituted by alkyl and/or halogen, C₁-C₆-haloalkyl, phenyl-C₁-C₄-alkyl, C₂-C₆-alkenyl or C₂-C₆-alkynyl;
R⁸ is hydrogen, C₁-C₆-alkyl or C₂-C₆-alkenyl; or
R⁷, R⁸ together with the nitrogen atom to which they are attached are a saturated or partially unsaturated 5-, 6- or 7-membered nitrogen heterocycle which may have 1 further heteroatom selected from the group consisting of O, S and NR¹⁰ as ring member and which may have 1 or 2 substituents selected from the group consisting of C₁-C₆-alkyl, C₁-C₆-haloalkyl, halogen and hydroxyl, where R¹⁰ is as defined in claim 1.

10. The compound according to claim 1 of the formula I in which R¹ is hydroxyl and one of the radicals Y or X is N.

11. The compound according to claim 1 of the formula I in which R¹ is halogen.

12. The compound according to claim 1 in which R² is hydroxyl, Y is C—R⁴ and X is C—R⁴ or N.

13. The compound according to claim 1 in which R² is halogen, C₁-C₆-alkyl or C₁-C₆-haloalkyl.

14. The use of a compound of the formula I 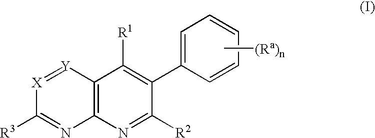
in which
X, Y independently of one another are N or C—R⁴;
n is 1, 2, 3, 4 or 5;
R^{a} is halogen, cyano, C₁-C₆-alkyl, C₁-C₆-alkoxy, C₁-C₆-haloalkyl, C₁-C₆-haloalkoxy, C₂-C₆-alkenyl, C₂-C₆-alkenyloxy or C(O)R⁵;
R¹ is halogen, cyano, C₁-C₆-alkyl, C₁-C₆-haloalkyl, C₂-C₆-alkenyl, C₂-C₆-alkynyl, C₃-C₈-cycloalkyl which is optionally mono- or polysubstituted by alkyl and/or halogen, C₅-C₈-cycloalkenyl which is optionally mono- or polysubstituted by alkyl and/or halogen, OR⁶, SR⁶ or NR⁷R⁸;
R² is halogen, cyano, C₁-C₆-alkyl, C₁-C₆-haloalkyl, C₂-C₆-alkenyl, C₂-C₆-alkynyl, C₃-C₈-cycloalkyl which is optionally mono- or polysubstituted by alkyl and/or halogen, C₅-C₈-cycloalkenyl which is optionally mono- or polysubstituted by alkyl and/or halogen, OR⁶, SR⁶ or NR⁷R⁸;
R³ is hydrogen, C₁-C₆-alkyl, C₁-C₆-haloalkyl or C₃-C₆-cycloalkyl which is optionally mono- or polysubstituted by alkyl and/or halogen;
R⁴ is hydrogen, halogen, C₁-C₆-alkyl, C₁-C₆-haloalkyl or C₃-C₆-cycloalkyl which is optionally mono- or polysubstituted by alkyl and/or halogen;
R⁵ is hydrogen, OH, C₁-C₆-alkyl, C₁-C₆-alkoxy, C₁-C₆-haloalkyl, C₁-C₆-haloalkoxy, C₂-C₆-alkenyl, C₁-C₆-alkylamino or di-C₁-C₆-alkylamino, piperidin-1-yl, pyrrolidin-1-yl or morpholin-4-yl;
R⁶ is hydrogen, C₁-C₆-alkyl, C₁-C₆-haloalkyl, phenyl-C₁-C₄-alkyl where phenyl may be mono- or polysubstituted by halogen, alkyl or alkoxy, C₂-C₆-alkenyl or COR⁹;
R⁷, R⁸ independently of one another are hydrogen, C₁-C₁₀-alkyl, C₂-C₁₀-alkenyl, C₄-C₁₀-alkadienyl, C₂-C₁₀-alkynyl, C₃-C₈-cycloalkyl, C₅-C₈-cycloalkenyl, C₅-C₁₀-bicycloalkyl, phenyl, phenyl-C₁-C₄-alkyl, naphthyl,
a 5- or 6-membered saturated or partially unsaturated heterocycle which may have 1, 2 or 3 heteroatoms selected from the group consisting of N, O and S as ring members, or
a 5- or 6-membered aromatic heterocycle which may have 1, 2 or 3 heteroatoms selected from the group consisting of N, O and S as ring members,
where the radicals mentioned as R⁷, R⁸ may be partially or fully halogenated and/or may have 1, 2 or 3 radicals R^{b}, where
R^{b} is selected from the group consisting of cyano, nitro, OH, C₁-C₆-alkyl, C₁-C₆-alkoxy, C₁-C₆-haloalkyl, C₁-C₆-haloalkoxy, C₁-C₆-alkylthio, C₂-C₆-alkenyl, C₂-C₆-alkenyloxy, C₂-C₆-alkynyl, C₂-C₆-alkynyloxy, C₁-C₆-alkylamino, di-C₁-C₆-alkylamino, piperidin-1-yl, pyrrolidin-1-yl or morpholin-4-yl;
R⁷ and R⁸ together with the nitrogen atom to which they are attached may also form a 5-, 6- or 7-membered saturated or unsaturated heterocycle which may have 1, 2, 3 or 4 further heteroatoms selected from the group consisting of O, S, N and NR¹⁰ as ring members, and may be partially or fully halogenated and which may have 1, 2 or 3 radicals R^{b}; and
R⁹, R¹⁰ independently of one another are hydrogen or C₁-C₆-alkyl;
or an agriculturally acceptable salt thereof for controlling phytopathogenic fungi.

15. A method for controlling phytopathogenic fungi, which comprises treating the fungi or the materials, plants, the soil or seed to be protected against fungal attack with an effective amount of a compound of the formula I according to claim 14 and/or with an agriculturally acceptable salt of I.

16. A composition for controlling phytopathogenic fungi, comprising at least one compound of the formula I according to claim 14 and/or an agriculturally acceptable salt of I and at least one liquid or solid carrier.

17. A ketone of the formula IIID 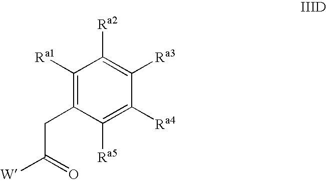
in which
W′ is C₁-C₆-alkyl, C₁-C₆-haloalkyl, C₂-C₆-alkenyl, C₂-C₆-alkynyl, C₃-C₈-cycloalkyl which is optionally mono- or polysubstituted by alkyl and/or halogen, C₅-C₈-cycloalkenyl which is optionally mono- or polysubstituted by alkyl and/or halogen;
R^{a1} is fluorine, chlorine, trifluoromethyl or methyl;
R^{a2} is hydrogen or fluorine;
R^{a3} is hydrogen, fluorine, chlorine, cyano, C₁-C₄-alkyl, C₁-C₄-alkoxy or C₁-C₄-alkoxycarbonyl;
R^{a4} is hydrogen, chlorine, or fluorine;
R^{a5} is hydrogen, fluorine, chlorine, C₁-C₄-alkyl or C₁-C₄-alkoxy.
